(19) United States
(12) Patent Application Publication
(10) Pub. No.: US 2007/0141637 A1
(43) Pub. Date: Jun. 21, 2007

(54) Marker for fenestrae

(76) Inventors: David T. Shima, Barnet (GB); Sofia Ioannidou, Boston, MA (US); Gregory S. Robinson, Wilmington, MA (US); Heming Xing, Newton, MA (US)

Correspondence Address: (OSI) EYETECH, INC., 41 PINELAWN ROAD, MELVILLE, NY, 11747, US

(21) Appl. No.: 11/274,800

(22) Filed: Nov. 15, 2005

Related U.S. Application Data
(60) Provisional application No. 60/628,085, filed on Nov. 15, 2004.

(51) Int. Cl. G01N 33/567; C12Q 1/37

(52) U.S. Cl. 435/7.2; 435/23

(57) ABSTRACT

The invention relates to a plasma membrane marker for identifying fenestrae. The invention also relates to a method of visualizing fenestrae utilizing a plasma membrane marker and light microscopy. The invention also relates to a method of identifying a plasma membrane marker for fenestrae. In particular, the invention relates to the characterization of moesin as a component of fenestrae sieve plates. More particularly, the invention relates to the use of moesin as a plasma membrane marker. Moesin may be used as a plasma membrane marker for the identification of fenestrae or permeability of endothelial cells.

### RELATED APPLICATION

This Application claims the benefit of U.S. Provisional Application No. 60/628,085, filed on Nov. 15, 2004. The entire teachings of the above application is incorporated herein by reference.

### FIELD OF THE INVENTION

The invention relates to a marker for fenestrae. The invention also relates to a method of visualizing fenestrae utilizing a marker and light microscopy. The invention also relates to a method of identifying a marker for fenestrae. More specifically the invention relates to the use of moesin as a marker for fenestrae.

### BACKGROUND OF THE INVENTION

Fenestrae are sub-endothelial structures, along with caveolae, transendothelial channels and vesiculo-vacuolar organelles that regulate transcellular permeability. Transcellular permeability is described as the passage of plasma components across endothelial cells. Precise regulation of blood-tissue interchange is critical for proper integration of organ physiology with the cardiovasculature. Accordingly, microvascular endothelial cells, the primary barrier to free blood-tissue exchange, adopt highly specialized features such as fenestrae to mediate and monitor the flux of macromolecules and fluids across the vascular wall.

Ultrastructural studies have described fenestrae as transcellular circular pores with an average diameter of ˜60 nm (though they can be as large as ˜125 nm within the liver sinusoidal endothelium). Fenestrae are encountered in the most attenuated regions of the endothelium, where the cell profile is as little as 40 nm, and span the entire thickness of the cell without disrupting the continuity of the cell membrane. The substances that traverse the pore do not encounter the contents of the cytoplasm, and are transported in a rapid and presumably energy-efficient manner. In most vascular beds, fenestrae contain a diaphragm composed of approximately 8 radial fibrils converging in a central knob, which further dissects the pore into 5-6 nm openings.

Fenestrae are known to occur in clusters of approximately 50-100, termed ‘sieve plates’, that are encircled by a microtubule-rich border. Within a sieve plate, fenestrae are typically found in a near linear arrangement with precise spacing between each pore.

Palade and the Simionescus pioneered the study of fenestrae composition in the 1960s, 70s and 80s by demonstrating that cationized ferritin (CF) preferentially deposited within a glycocalyx visible on the luminal aspect of the fenestral diaphragm. Recent studies identified an endothelial cell-specific protein, Plasmalemmal Vesicle 1 Protein (PV-1) as the first known component of the fenestral diaphragm. PV-1 is a 60 kDa Type II transmembrane glycoprotein that is believed to form homodimers that constitute the primary structural component of the diaphragm. PV-1, and the diaphragm are not unique to fenestrae. PV-1, and the diaphragm also reside within endothelial cell caveolae and transendothelial channels. Cross-linking studies in cells rich in caveolae were unable to identify new proteins associated with PV-1 (Stan, R. V., Am. J. Physiol. Heart Circ. Physiol., 2004. 286(4): p. H1347-53).

Fenestrae sieve plates are specialized membrane structures, whose molecular composition has attracted considerable interest, but in the absence of appropriate study tools, remained elusive.

### SUMMARY OF THE INVENTION

The invention is based, in part, upon the discovery of novel components of fenestrae sieve plates and novel components related to fenestrae formation and function. In particular the invention is based on the discovery of a marker for fenestrae sieve plates.

In one aspect, the invention relates to a method of identifying a marker for fenestrae sieve plates. In one embodiment the method comprises proteomics. In another embodiment, the method comprises evolutionary genomics. In one embodiment the invention relates to a method of characterizing moesin as a component of fenestrae sieve plates. In another embodiment the invention relates to a method of characterizing paralemmin as a component of fenestrae sieve plates or as a component of the process of forming fenestrae.

In another aspect, the invention relates to a marker for use in identifying fenestrae. In one embodiment, the marker is a plasma membrane marker for identifying fenestrae. In one embodiment the invention relates to moesin as a plasma membrane marker for use in identifying fenestrae. In one embodiment the invention relates to paralemmin as a marker for use in identifying fenestrae.

In another aspect, the invention relates to a method of using a marker to identify fenestrae. In one embodiment the invention relates to a method of using moesin as a marker to identify fenestrae. In another embodiment the invention relates to a method of using paralemmin as a marker to identify fenestrae.

In another aspect, the invention relates to a method of visualizing fenestrae using a marker and light microscopy. In one embodiment, the invention relates to a method of visualizing fenestrae using moesin as a marker and light microscopy. In another embodiment, the invention relates to a method of visualizing fenestrae using a marker combination for fenestrae and light microscopy. In one particular embodiment, the marker combination comprises moesin and PV-1.

Moesin can be useful because of its immunoreactivity, in probing for non-diaphragmed capillary beds, which lack PV-1, but contain moesin. Moesin can also be useful, because of its specificity for fenestrae, for rendering the classification of fenestrae less ambiguous on the basis of immunoreactivity for PV-1, a marker with dual specificity for caveolae and fenestrae in combination with anti-moesin labeling. Moesin can also be useful as an indicator of vascular endothelial growth factor (VEGF) dependency and as an indicator of fenestrae or permeability in an endothelial cell. Moesin can also be useful as a pathological therapeutic target.

The connection between the membrane-cytoskeleton adaptor moesin and fenestrae bears significant functional implications, and opens new avenues in the study of fenestrae's biogenesis. The discovery of moesin as a novel component of fenestrae is an important step towards the visualization of fenestrae by light microscopy.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 is a schematic of the cationic colloidal silica isolation protocol. Cells are coated on the dish with a solution of cationic colloidal silica, which is neutralized by a subsequent coating with sodium polyacrylate. Cells are then lysed in a hypotonic buffer using a dounce homogenizer, and the various subcellular components separated by centrifugation. A low-speed pellet containing nuclei and silica-coated plasma membranes is then fractionated by an ultracentrifugation step through a nycodenz gradient to obtain a pellet of silica-coated plasma membranes.

FIG. 2 shows a biochemical analysis of cationic colloidal silica plasma membrane isolation from bEND5 (A) and Py4.1 (B) cells. 10 μg of protein from each fraction was subjected to SDS-PAGE electrophoresis and Western Blotting using antibodies to the indicated proteins. Enrichment for the plasma membrane markers PV-1 and VE-cadherin, and depletion for the Golgi marker GM130 is seen in the plasma membrane fractions of both bEND5 and Py4.1 cells.

FIG. 3 shows a TEM examination of plasma membrane pellets from untreated (A) and latrunculin-treated (B) endothelial cells. Silica coated plasma membrane sheets are the predominant structure in both pellets. Bar=2 μm.

FIG. 4 shows a TEM examination of silica-isolated plasma membrane pellets at higher magnifications. (A) Cross-section through a whole cell contaminant of the plasma membrane pellet. (B) Caveolae are isolated intact with the plasma membranes of control endothelial cells. (C) and (D) High-magnification images of plasma membranes from latrunculin-treated endothelial cells show the silica masking all underlying membrane ultrastructure.

FIG. 5 shows the quality control of isolated plasma membranes prior to proteomic analysis by immunofluorescence (A) and western blotting (B). The success of fenestrae induction was monitored by the characteristic staining of PV-1 for fenestrae patches in areas that exclude microtubules shown in (A) for a bEND5 cell. Anti-PV-1 labeling (i), anti-tubulin labeling (ii), and the overlay (iii) are shown. (B.)The success of the isolation protocol was monitored by enrichment for PV-1 and de-enrichment for GS-28 in plasma membrane and nuclei (PM+nu), and plasma membrane (PM) fractions versus whole cell homogenates of control (C) and latrunculin induced (L) Py4.1 (a) and bEND5 (b) cells. In bEND5 cells the additional control sampled at time 0 (C1) can be distinguished from the vehicle-induced sample (C2).

FIG. 6 shows representative 2D-gels from vehicle—induced control (A and C) and latrunculin-induced (B and D) samples from the bEND5 (A and B) and Py4.1 (C and D) cells.

FIG. 7 shows spot intensity calculation using Progenesis Software. (A) shows spot 659 from the Py4.1 cell analysis, recognized as being 1.772 fold higher in the latrunculin-induced samples (520b, 550b, 561b) compared to control samples (480a, 396a, 560a, 508a). (B) shows spot 401 from the Py4.1 cell analysis, which although recognized as being 3.8 fold higher in the latrunculin-induced samples, is actually part of a train of spots characteristic of different phosphorylation states of the same protein.

FIG. 8 shows the categorization of candidate proteins with respect to their subcellular localization (A) and function (B). For proteins falling into more than one category, the value was split between them. Where multiple isoforms or polypeptide chains for the same protein were identified, the protein was counted only once.

FIG. 9 shows images representing validation of candidate proteins by Western Blotting. Equal amounts of protein from CPM (control cells' plasma membranes) and LPM (latrunculin-induced cells' plasma membranes) were separated by SDS-PAGE electrophoresis and probed for the indicated proteins by Western Blotting. Targets from the bEND5 cell analysis are shown in (A) and from the Py4.1 cells analysis in (B). Enrichment in induced cells is confirmed for phosphorylated moesin, hnRNP K, cofilin, and alpha-enolase. A comparison of the enrichment between the membrane fractions of control and induced cells (CPM, LPM) to the enrichment between the whole cell—homogenates of control and induced- cells (CW, LW) for alpha-enolase and cofilin is shown in (C). The bands for internal membranes (CIM, LIM), and for plasma membrane and nuclei (CPM+N, LPM+N) are also indicated for cofilin.

FIG. 10 shows images representing validation of candidate proteins by Immunofluorescence. Induced bEND5 cells were stained for PV-1 (A, D, G, J) and the following candidate proteins: cofilin (B), annexin (E), radixin (H), and moesin (K). The corresponding overlays (C), (F), (I), (L) show that radixin and moesin localize on the same patches that are labeled by PV-1, whereas cofilin and annexin are excluded from those regions.

FIG. 11 shows ERM family distribution within fenestrae patches. Induced bEND5 cells were immunostained for moesin (A), ezrin (D), merlin (G), and radixin (J) and counterstained with anti-PV-1 (B, E, H, K), to mark the location of fenestrae. Overlays of the antibody combinations are shown in C,F,I,L. Moesin (A) and radixin (J) appear to co-localize with the PV-1 staining on fenestrae (B,C) and (K,L), respectively, while ezrin (D) and merlin (G) appear excluded from the PV-1 positive regions of the cell (E,F) and (H,I), respectively. (Bar=20 μm)

FIG. 12 shows distribution of ERM family members in vivo. Immunohistochemistry on mouse eye sections was performed using antibodies against moesin (A,E), ezrin (B,F), and PV-1 (C,G). Moesin colocalizes with PV-1 in the choriocapillaris, while ezrin is confined to the retinal pigment epithelium layer, as seen in the low (D) and high (H) magnification overlays. Some coincidence between moesin and ezrin is seen in the sclera, below the choriocapillaris.

FIG. 13 is a table summarizing samples used for proteomic analysis.

FIG. 14 is table of protein identity assignments for spots in each sample from subtractive proteomic analysis.

FIG. 15 is a table summarizing intensities of spots selected and excised for mass spectrometry, with their accompanying fold differences and p-values.

FIG. 16 is a table summarizing candidate proteins grouped according to their function. Columns L vs C2 (1), L vs C2 (2), and L vs C2 (3) refer to fold change in spot intensity in individual batch analyses performed for bEND5 cell experiments 1, 2, and 3 respectively. Highlighted are the fold changes that determined the selection of the particular spot for excision and subsequent protein content identification.

FIG. 17 is a Venn Diagram illustrating comparative genomic approach toward fenestrae-specific genes. The Ven Diagram compares the Human, Xebrafish and Arabidopsis genomes.

FIG. 18 is a table showing a genetic comparison of fenestrated and non-fenestrated organisms. The table compares the number and percentage of genes compared to the number of human genes.

FIG. 19 is a table showing the proteins that were identified by both comparative genomics and in proteomics.

FIG. 20 illustrates a study relating the dominant negative form of moesin (N-moesin/GFP) to sieve plate composition. FIG. 20A-C show high-expressing cells forming sieve plates lacking endogenous moesin. FIG. 20D-F show high-expressing cells forming sieve plates lacking endogenous. FIGS. 20A, 20D, 20G and 20J show N-moesin/GFP was confined to areas of the cell membrane that were morphologically similar to sieve plates. FIG. 20J-L shows the sieve plates occurred in thin areas of the cell periphery, and were distinct from the microtubule- and organelle-rich border that encircled them. FIG. 20G-I shows the expression of PECAM throughout the cell surface was unchanged. FIG. 20M shows that in high-expressing induced cells, GFP was distributed throughout the cell cytoplasm, with a preference for microtubule- and organelle-rich regions lying between sieve plates. FIG. 20M-O shows sieve plates presented a regular composition, containing PV-1, moesin and the discrete network of filamentous actin.

### DETAILED DESCRIPTION OF THE INVENTION

The present invention relates to a method of determining the identity of components of fenestrae. The present invention also relates to a method of determining the identity of components involved in the formation and finction of fenestrae. In particular the invention relates to a method of identifying molecules that constitute the rim of the pore, or the fenestrae sieve plate. Fenestrae induction, is believed to involve a recruitment of novel components to the plasma membrane.

In one aspect, the invention provides a method for identifying a marker for fenestrae in an endothelial cell line comprising an in vitro fenestrae induction and a sub-cellular proteomic analysis. In particular, the invention provides a method of identifying a marker for fenestrae in an endothelial cell line utilizing subtractive proteomic analysis.

In one embodiment, the invention provides a method of identifying a marker for fenestrae in an endothelial cell line comprising the steps of:
a) isolating plasma membranes from a batch of fenestrae induced endothelial cells and a batch of fenestrae un-induced endothelial cells.
b) running a two-dimensional electrophoresis gel on material isolated from the batch of fenestrae induced endothelial cells;
c) running a two-dimensional electrophoresis gel on material isolated from the batch of fenestrae un-induced endothelial cells;
d) determining fold differences between staining intensities of spots in the two-dimensional gel of the batch of fenestrae induced endothelial cells and the spots in the two-dimensional gel of the batch of fenestrae un-induced endothelial cells;
e) excising a spot; and
f) analyzing the spot against a database of fingerprints from theoretical tryptic digests of proteins so as to assign a particular protein identity to the spot.

In another embodiment, the step of isolating plasma membranes comprises a silica isolation procedure.

In another embodiment, the method further comprises the step of producing a characteristic mass fingerprint for the excised spot utilizing mass spectrometry.

In another embodiment, the method further comprises the step of confirming the assigned protein identity of the spot. In one particular embodiment, the step of confirming the protein identity of the spot comprises immunoblotting antibodies onto the relevant fractions of candidate proteins. In another particular embodiment, the step of confirming the findings of the spot comprises immunofluorescence against a characteristic pattern of PV-1 in induced cells as a reference standard. In another particular embodiment, the step of confirming the findings of the spot comprises light microscopy.

In another aspect, the invention provides a method for identifying a marker for fenestrae in an endothelial cell line comprising evolutionary genomics (comparative genomics). With completed genomic sequences available for multiple species, a comparative genomics approach is used to identify genes that are specifically present in fenestrated organisms. Genes identified from comparative genomics are compared to the protein identified from proteomics. Common represent gene targets that are highly likely to be involved in the composition or process of forming fenestrae.

In one embodiment, the endothelial cell line is a brain endothelioma cell line. In another embodiment, the endothelial cell line is selected from the group consisting of a bEND5 endothelial cell line and a Py4.1 endothelial cell line. In one particular embodiment, the endothelial cell line is a bEND5 endothelial cell line.

Another aspect of the present invention relates to a method of visualizing fenestrae utilizing light microscopy.

The present invention also relates to a method for visualizing fenestrae in an endothelial cell line, wherein the method comprises the steps of:
a) staining an endothelial cell line with an anti-moesin antibody;
b) imaging the endothelial cell line utilizing a light microscope; and
c) visualizing fenestrae based on the level of the fluorescently labeled anti-moesin bound to moesin in the endothelial cell line.

In one embodiment, the anti-moesin antibody is fluorescently labeled.

The present invention also relates to a plasma membrane marker for detecting fenestrae. In one aspect, the invention relates to a marker for detecting fenestrae in an endothelial cell line. In another aspect, the invention relates to a marker for detecting fenestrae in vivo.

In one embodiment, the marker for detecting fenestrae comprises a protein selected from the group consisting of moesin, paralemmin, radixin, cofilin, twinfilin, alpha-enolase, annexin II and hnRNP K, musculin, putative RNA-binding protein 3, and nucleoside diphosphate kinase B. In one particular embodiment, the plasma membrane marker for detecting fenestrae comprises moesin. In one particular embodiment, the marker for detecting fenestrae comprises paralemmin.

The present invention also relates to a plasma membrane marker combination for detecting fenestrae, wherein the plasma membrane marker combination comprises PV-1 and moesin.

The present invention also relates to methods of using moesin as a plasma membrane marker to identify fenestrae. In one embodiment the invention relates to a method of using moesin as a plasma membrane marker to identify fenestrae in an endothelial cell line. In another embodiment the invention relates to a method of using moesin as a plasma membrane marker to identify fenestrae in vivo.

One embodiment of the invention provides a method of using moesin as an indicator of fenestrae in an endothelial cell line. Another embodiment of the invention provides a method of using moesin as a marker for permeability in an endothelial cell line. Another embodiment of the invention provides a method of using moesin as a pathological therapeutic target in an endothelial cell line. Another embodiment of the invention provides a method of using moesin as an in vivo pathological therapeutic target.

In one embodiment, the first step of the sub-cellular proteomic analysis includes isolating plasma membranes from other parts of the cell. The isolation of plasma membranes eliminates the non-specific effects of the induction, to minimize the protein complexity of the mixture to be analyzed, and to focus on the subcellular fraction where the relevant changes occur.

In one embodiment, the step of isolation of plasma membranes uses an affinity purification method based on the physico-chemical properties of the plasma membrane as a whole. The method uses the high affinity of cationic colloidal silica for negatively charged plasma membranes. Upon binding of silica, plasma membranes acquire a unique density that can be used to separate them from the remainder of the cell (Jacobson, B. S. and D. Branton, Science, 1977, 195(4275): p. 302-4. Chaney, L. K. and B. S. Jacobson, J Biol Chem, 1983. 258(16): p. 10062-72; Stolz, D. B. and B. S. Jacobson, J Cell Sci, 1992. 103 (Pt 1): p. 39-51; Mason, P. W. and B. S. Jacobson, Biochim Biophys Acta, 1985. 821(2): p. 264-76). Silica can be attracted to fenestrated areas of the membrane due to their particularly high concentrations of negative charge. Silica can also stabilize the membrane substructure by firmly holding the negative charges in place (Simionescu, N., M. Simionescu, and G. E. Palade, J Cell Biol, 1981. 90(3): p. 605-13.). Silica-coating is reported to prevent vesicularization of the fragmented plasma membrane, and to maintain spatiotemporal protein and lipid relationships without modifying any of the components as silica is chemically inert (Schnitzer, J. E., et al., Science, 1995. 269(5229): p. 1435-9; Schnitzer, J. E., et al., Proc Natl Acad Sci USA, 1995. 92(5): p. 1759-63). The method of cationic colloidal silica isolation technique results in significant plasma membrane enrichment over the whole cell homogenate.

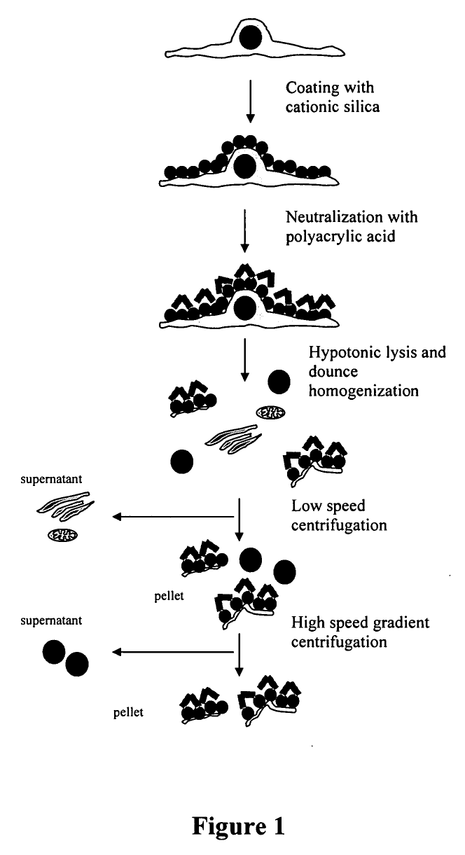

An example of the cationic colloidal silica plasma membrane isolation was applied to latrunculin-induced and vehicle-induced bEND5 and Py4.1 cell cultures, and is schematically depicted in FIG. 1.

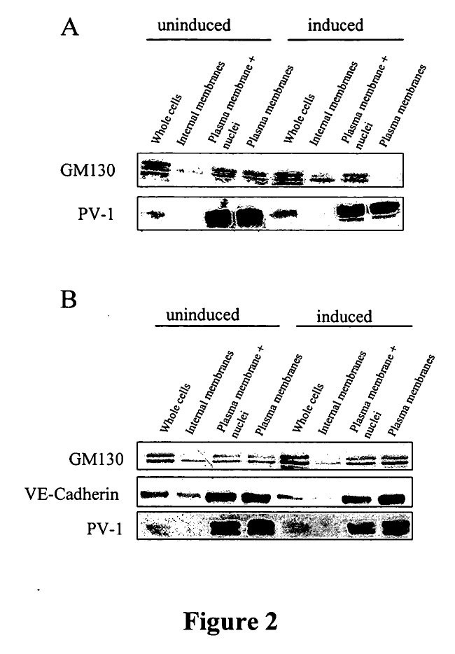

The progress of the isolation procedure was followed biochemically by retaining samples from various steps in the procedure and monitoring for the presence of particular organelle markers after SDS-PAGE electrophoresis and western blotting (FIG. 2). A comparison of equal amounts of proteins for whole cell, internal membrane, plasma membrane and nuclei, and plasma membrane fractions showed a progressive enrichment for plasma membrane markers such as VE-cadherin and PV-1. Enrichment for VE-cadherin and PV-1 in the plasma membrane fraction versus whole cells was between about 5 and 40 fold, while golgi markers such as GM130 showed a depletion between about 2 and 20 fold. In addition, no signal for plasma membrane markers was detected in the internal membrane fraction, suggesting that there is no or minimal loss of plasma membranes in the supernatant of the first low-speed centrifugation step, and plasma membranes co-sediment with nuclei as predicted due to their unique new density.

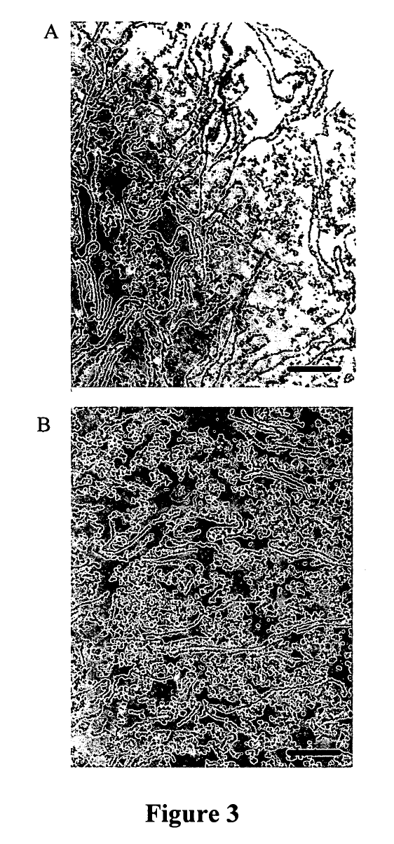

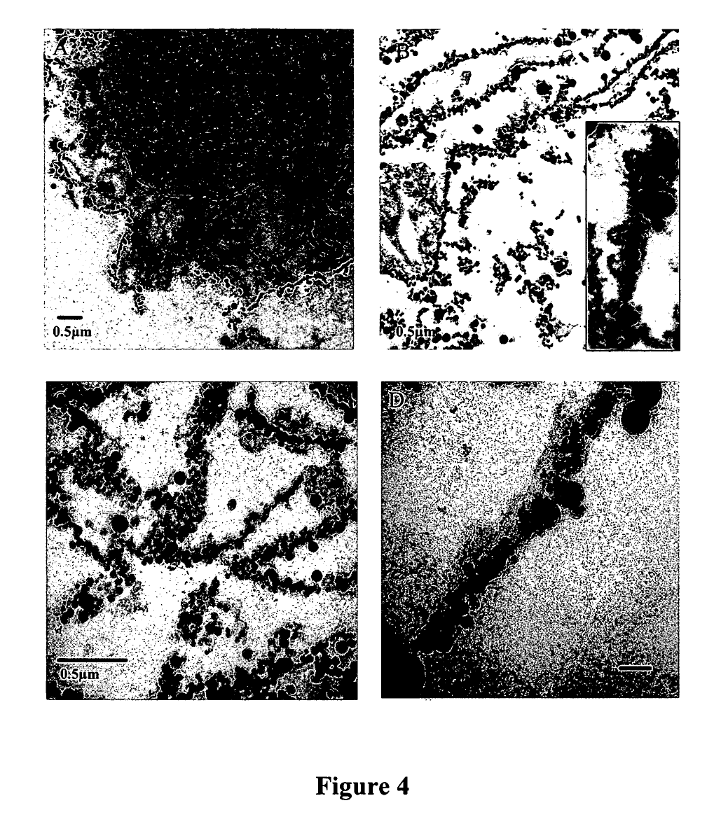

The silica isolation procedure was validated ultra-structurally by examining thin sections from fixed plasma membrane pellets using TEM. Low magnification images from plasma membrane pellets of uninduced and induced bEND5 cells showed that silica-coated plasma membrane sheets, appearing as ‘beads on a string’, were the predominant element of both fractions (FIG. 3). Occasionally whole cells were encountered in the pellets (FIG. 4A), consistent with the biochemical data and the light microscopy observations. High magnification images of plasma membranes from uninduced cells revealed the presence of caveolae attached to the cytoplasmic aspect of the isolated membrane sheets (FIG. 4B). Both caveolae, and intracellular organelles in whole cells appeared morphologically intact, and the silica was confined to the exterior of the cell. The size of the silica particles had an average diameter of 20-50 nm and therefore masked any underlying ultrastructure of the membrane itself, including fenestrae (FIGS. 4C and 4D).

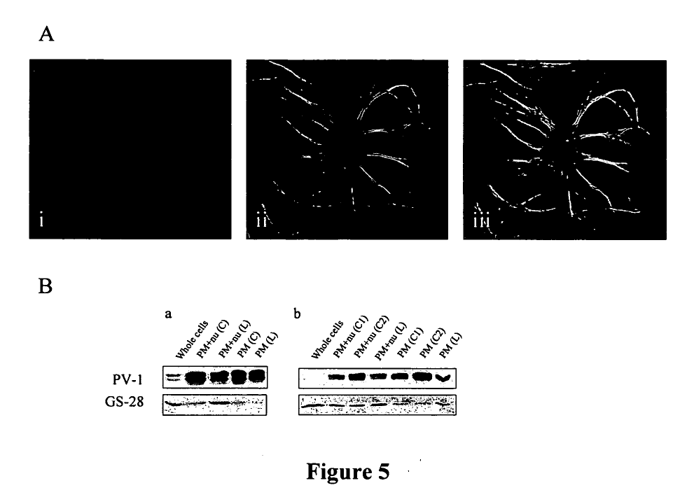

To ensure the quality of the material processed for a proteomic comparison, each induction was verified by light microscopy and each purification was followed biochemically. Coverslips included in the culture dishes were fixed at the start of the isolation protocol and immunostained for the diaphragm marker PV-1 and microtubules. PV-1 staining in thin areas of the cell that were devoid of cytoskeleton, caveolae, or intracellular organelles represented fenestrae ‘sieve plates’. The characteristic fenestrae patches were apparent as closely apposed ‘islands’ separated by tubulin ‘spacers’ in induced bEND5 and Py4.1 cells (FIG. 5A).

For each cell type, latrunculin-induced and vehicle-induced material was obtained for comparison. For bEND5 cells, the additional control of cells entirely untreated (referred to as C1), and processed for plasma membrane isolation at the start of the induction (time 0) was used in order to be able to pinpoint exactly any differences due to the vehicle. Aliquots of the various subcellular fractions were retained throughout the progress of the purification protocol and examined for the enrichment of PV-1 and the depletion of intracellular components such as the golgi marker GS28 (FIG. 5B).

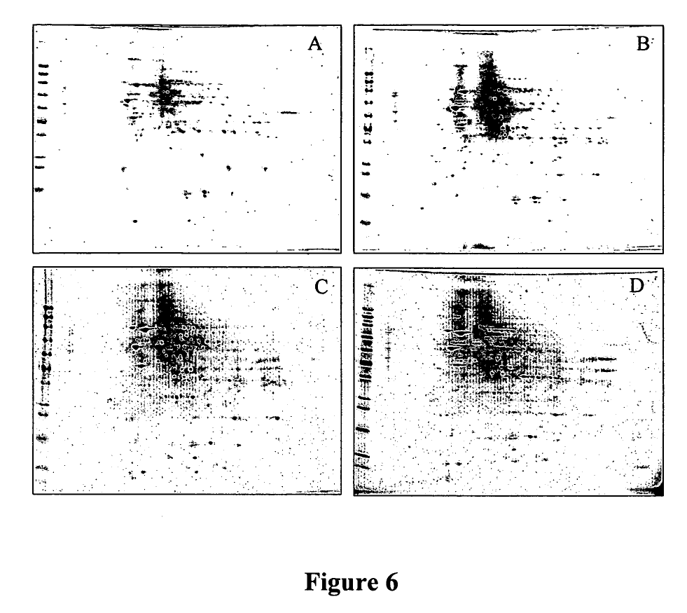

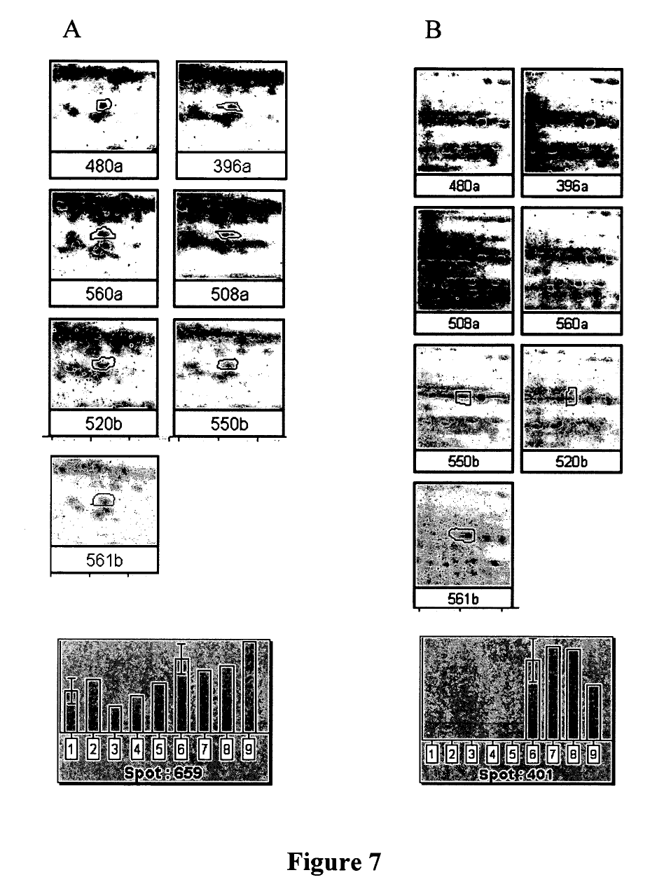

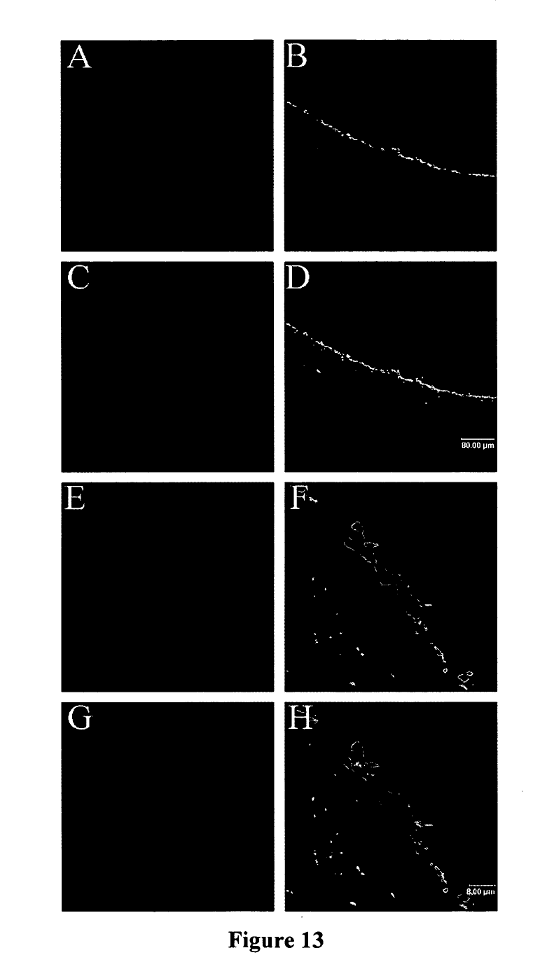

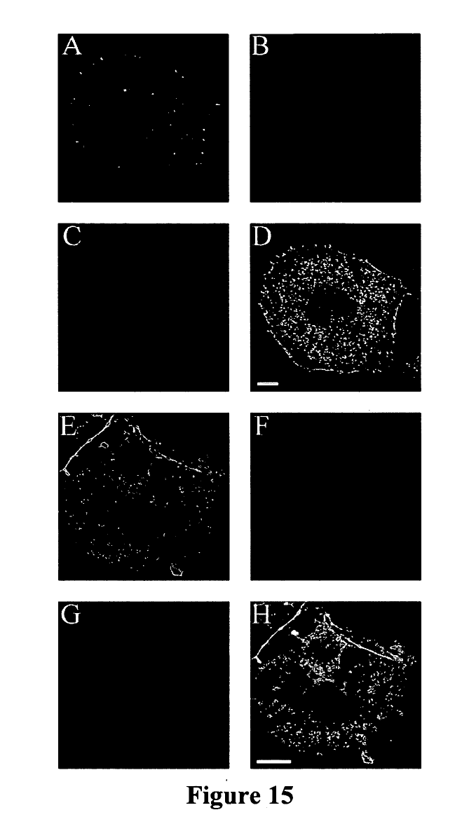

In one embodiment the method of identifying components of fenestrae comprises the step of Subtractive 2D-gel electrophoresis analysis. Sufficient material was pooled to generate one batch of triplicate gels for vehicle-induced and un-induced Py4.1 cells, and three sets of triplicate gels for untreated, vehicle-induced, and induced bEND5 cells (FIG. 6 and FIG. 13). All gels per cell type were analyzed collectively using ProGenesis® software (Prolific, Inc. Newark, Calif.) to obtain fold differences between the staining intensities of spots in the induced and the uninduced samples and p-values for those differences using the Students t-test (FIG. 7). In the first round of analysis, spots that qualified for one or more of the following criteria were selected: a) fold increase in staining intensity greater than 2 in the induced sample, b) p-value less than 0.05 for any fold increase, c) spot being unique in induced sample. A summary of all the spots selected for mass spectrometry, with their accompanying fold differences and p-values is shown in FIG. 15.

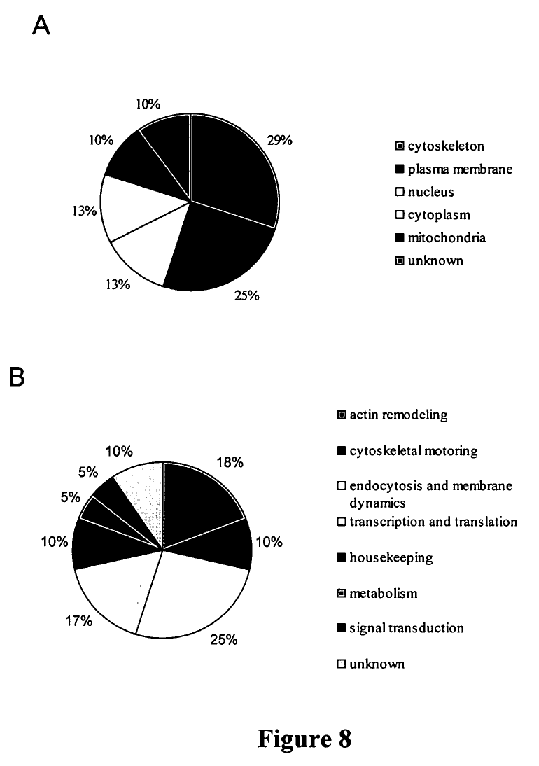

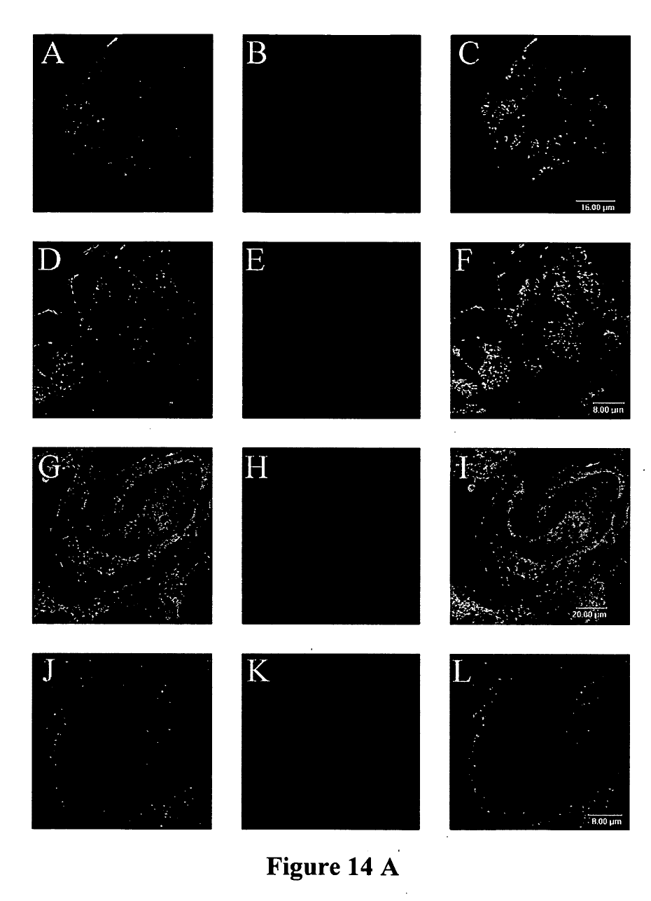

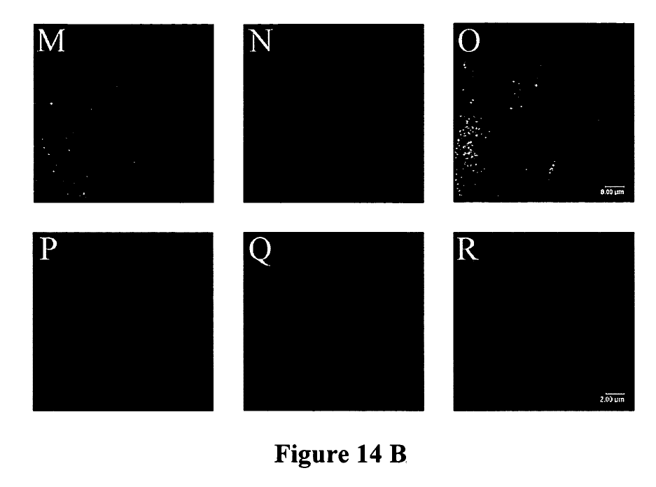

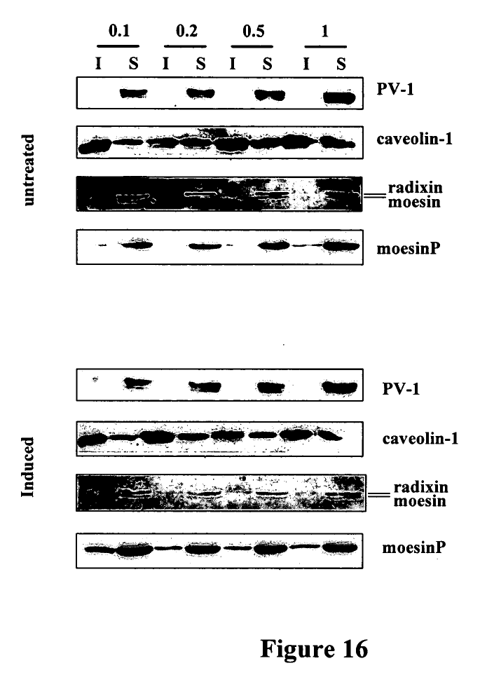

In one embodiment the method of identifying components of fenestrae comprises the step of assigning identity using mass spectrometry. Excised spots were digested with trypsin and analyzed by Mass Spectrometry to produce a characteristic peptide mass fingerprint for each spot, provided that sufficient protein amounts were present. Each mass fingerprint was then matched against a database of fingerprints from theoretical tryptic digests of proteins so as to assign a particular protein identity to each spot. Information such as the percent coverage of a theoretical protein identity by the tryptic fragments obtained, the fraction of tryptic fragments matching that identity, the MOWSE score, and the relationship between the pI and molecular weight of the excised spot and that of the theoretical identity, were all considered prior to assigning a particular identity. If more than one identity was assigned, the identity can be explained either by similarities in tryptic fragment patterns between potential identities, or by the presence of more than one protein within a single spot on the gel. In addition, separate spots frequently corresponded to the same protein, especially when found in clusters representing collections of post-translational states of a given protein. Assigned protein identities for spots in each sample are presented in detail in FIG. 14, and summarized according to their function in FIG. 16. FIG. 16 is a table summarizing candidate proteins grouped according to their function. For proteins with more than one function, only the best-known one is indicated. Fold change and accompanying p-value is shown for all types of analysis performed. Total change refers to the fold change in spot intensity in induced versus control cells across all gels in a sample. Columns L vs C2 (1), L vs C2 (2), and L vs C2 (3) refer to fold change in spot intensity in individual batch analyses performed for bEND5 cell experiments 1, 2, and 3 respectively. Note that more than 1 protein identity can be assigned to one spot (e.g. bEND5 400), and the same protein identity can be encountered in multiple spots (e.g. bEND5 526 and 527). Highlighted are the fold changes that determined the selection of the particular spot for excision and subsequent protein content identification. The categories in which candidates fall, with respect to their subcellular localization or function are illustrated in pie-charts in FIG. 8.

In one embodiment the method of identifying components of fenestrae comprises the step of target validation. In one particular embodiment, target validation comprised immunoblotting for candidate proteins. In another particular embodiment, target validation comprised immunofluorescence.

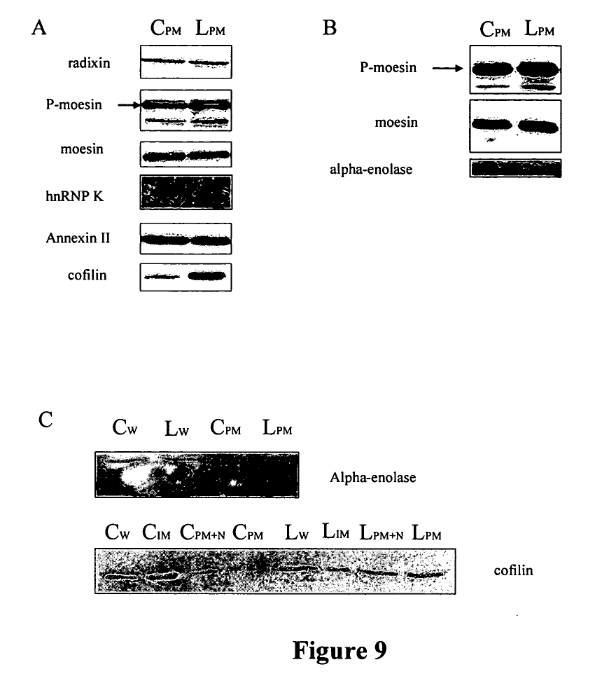

To confirm the findings of the subtractive proteomic analysis, immunoblotting on the relevant fractions was performed with antibodies to candidate proteins. By comparing equal amounts of protein in the two conditions, change in the protein levels of cofilin, alpha-enolase, radixin, moesin, annexin II, and hnRNP K was evaluated (FIG. 9A, 9B).

Cofilin was upregulated in all induced membrane fractions examined. Annexin II was variably upregulated. Alpha-enolase and hnRNP K appeared both to be upregulated. Probing with an antibody that primarily recognizes the phosphorylated version of moesin, shows an increase in the phosphorylated version of moesin. The 2d-gel spot pattern for moesin appears as part of a ‘train of spots’ (FIG. 7) and is characteristic of proteins that exist in multiple phosphorylation states.

Consistent with the hypothesis of plasma membrane enrichment being necessary to unmask relevant differences between the samples, cofilin enrichment is only evident in the plasma membrane fraction of induced versus uninduced cells, while is absent in the comparison of whole cell homogenates (FIG. 9C). A similar pattern is seen for the primarily cytosolic protein alpha-enolase, where a higher portion of the protein co-purifies with the plasma membrane fraction in induced versus uninduced cells (FIG. 9C). In addition to the 48 KDa band of alpha-enolase, a 35 KDa band appears consistently only in plasma membrane fractions of control and induced cells, in both the bEND5 and Py4.1 samples.

Targets validated by western blotting were subsequently examined for their subcellular localization in relation to fenestrae by immunofluorescence on bEND5 cells. The characteristic pattern for PV-1 in induced cells was used as a reference standard for the presence and appearance of fenestrae.

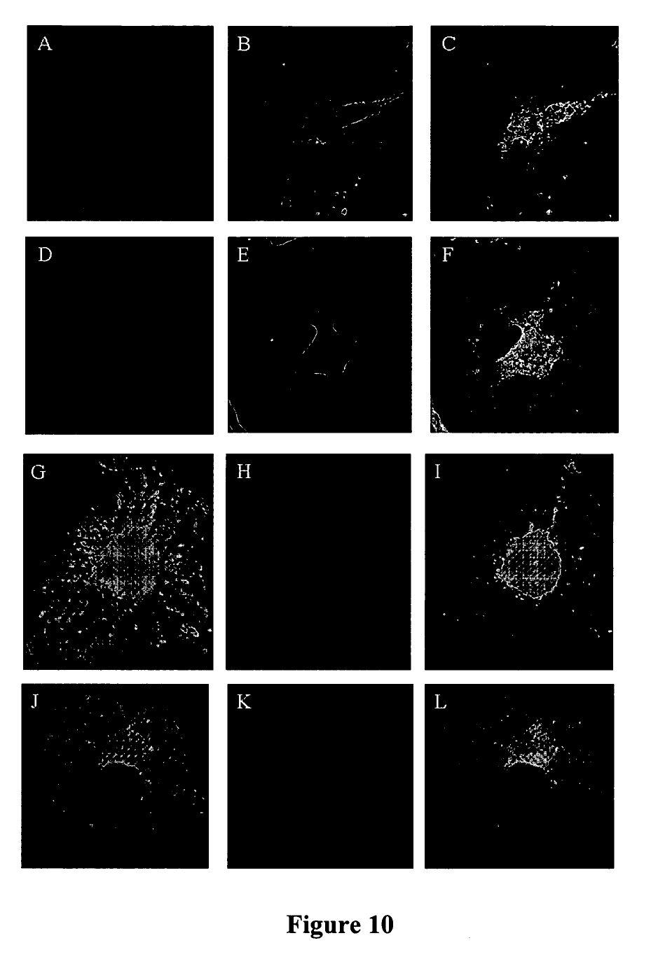

The actin binding protein cofilin and annexin II appeared to be associated with the thick areas of the cell that act as spacers between fenestrae patches (FIG. 10A-F). Alpha enolase was concentrated mainly in the perinuclear region in both control and induced cells. The membrane-cytoskeleton adaptors moesin and radixin are redistributed into the same fenestrae patches marked by PV-1 in induced cells (FIGS. 10C, 10D). The staining pattern obtained with goat polyclonal antibodies against moesin and radixin almost entirely overlapped with that for PV-1, and was confirmed for moesin using a mouse monoclonal antibody against it.

Subtractive proteomic analysis is advantageous because it yields a smaller and more manageable set of candidate proteins than high-resolution whole proteome analyses. In such a differential expression approach, the size of the candidate set can vary depending on the level of differences between the samples under comparison, and the stringency criteria set by the investigator. In this comparison of identical cell types differing in their treatment with an actin-disrupting agent, only spots that were either unique, greater than 2-fold upregulated in the treated sample, or increased in the treated sample with a confidence interval of p<0.05 were considered, and a set of 26 unique protein identities was obtained. Often, the same spot yielded more than one identity, which can be attributed to the non-absolute method of identity assignment used, based on matching mass fingerprints rather than amino acid sequences against protein databases. When the ambiguity between two or more identities could not be resolved, they were all deemed possible, taking into account the limitations in the identity assignment method and the resolving capabilities of the 2d-gel itself.

Overall, out of 26 protein identities, 23 were characterized gene products, 2 had been previously identified in functional screens (Ras suppressor protein 1, interferon-activatable protein 205), and one was partially characterized on the basis of structural similarity to known proteins (putative RNA-binding protein 3). Categorization of proteins on the basis of their reported, or in some cases putative, subcellular localization indicated that a total of 54% was associated with the plasma membrane and the co-purifying cytoskeleton, and another 33% with various other cytoplasmic structures. Within the set of plasma membrane associated proteins, none of the proteins had membrane spanning regions, and instead were peripherally attached or lipid anchored to the bilayer. Categorization of proteins with respect to their finction, indicated that the majority of candidates were involved in membrane remodeling or endocytic processes, while a great proportion was also occupied by actin binding proteins, cytoskeletal motors, and proteins with roles in transcription or translation.

When viewed in the context of fenestrae, candidate proteins can be split into two categories: those of structural components, and those involved in some stage of fenestrae biogenesis. The role of cytoskeletal remodeling in fenestrae formation has been documented. [24-28] Cofilin binds to both monomeric and filamentous actin and is reported to increase actin filament disassembly by depolymerising filaments from their pointed ends and by inhibiting spontaneous nucleotide exchange on monomeric GDP-bound actin [29, 30]. Twinfilin (or protein tyrosine kinase 9) has an ability to sequester actin monomers and prevent their nucleotide exchange is believed to inhibit monomer assembly [30, 31]. Transgelin is the least characterized of the actin binding protein candidates, but reportedly plays a role in cross-linking actin filaments into aggregates [32, 33], in a seemingly opposite role to that of cofilin and twinfilin. These endogenous actin binding proteins can have roles in the actin-filament disassembly that is required for fenestrae formation, as aids to the exogenously added latrunculin A, which presumably drives the process.

Myosins and kinesins are molecular motor proteins that move on actin-filaments or microtubules, respectively, performing work in a cell. Both are found as oligomers of heavy and light chains, with heavy chains serving as motor domains and light chains having either regulatory roles, in the case of myosins, or determining subcellular localization and cargo selection in kinesins [34-36]. Although only light chains were identified in this screen the entire protein functional unit is considered. Myosins and kinesins could catalyze this process by driving vesicles and organelles to or away from the area that is about to become fenestrated. Latrunculin A is reported to induce the internalization of caveolae and their movement along microtubules. [37]

Apart from cytoskeletal disassembly and organelle movement, a part of fenestrae biogenesis includes the remodeling of the membrane itself, from a continuous structure to that of a discontinuous one, intermitted by numerous pores. Endocytosis, as a means of membrane cycling from the plasma membrane to some internal organelle is a plausible mechanism for such membrane remodeling, and in this context, the tumor promoter Nm23, has been shown to be necessary for dynamin-mediated endocytosis of adherens junctions [39]. Paralemmin is another protein implicated in plasma membrane dynamics, through its association with the cytoplasmic face of membranes at sites of plasma membrane activity and its ability to induce cell expansion and process formation [40]. Radixin and moesin, representing the ERM (ezrin-radixin-moesin) family of proteins, can link the fundamental processes of actin disassembly and plasma membrane remodeling through their role as membrane-cytoskeleton adaptors [41]. The candidate proteins were validated both as upregulated using western blotting, and as being part of the fenestrae sieve plate by immunofluorescence.

In a mass-spectrometry based study of focal adhesion components, the identification of RNA binding proteins such as hnRNPs came as a surprise. In the same study, validation of these candidate proteins by immunofluorescence, highlighted a novel function as components of spreading initiation centers at the initial stages of focal adhesion assembly [44]. In the course of fenestrae biogenesis, attachments to the substratum also undergo remodeling. The absence of the typical vinculin staining that is characteristic of focal adhesions from fenestrated cells suggests that focal adhesions need to be disassembled. The candidate protein hnRNP K could participate in such a process that would essentially be the reverse of its newly reported function.

Independent confirmation for a number of candidate proteins identified in this study came from a report concerning the proteome of some small exocytic vesicles termed exosomes [45]. The presence of the diaphragm protein PV-1, along with myosin, ERM proteins, kinesin, alpha-enolase, translation initiation factor, and annexin II, in a list of 38 identified proteins is suggestive of a relationship between them in a similar structural context to that of fenestrae.

This study was the first to use a subtractive proteomic comparison of organelle—rich to organelle—deficient states of the same cell, in order to define such an organelle's protein complement. This study was also the first successful attempt to discover and validate proteins of the fenestrae sieve plate. In a similar fashion to PV-1, however, moesin and radixin are not unique to fenestrae, and form part of various other cell structures. The identified connection between ERM proteins and fenestrae can serve as a basis for expanding our knowledge.

Moesin and radixin, two of the newly identified components of fenestrae sieve plates described herein, are members of the ERM (ezrin-radixin-moesin) family of proteins, which are known to act as adaptors between the cortical cytoskeleton and the plasma membrane [66]. The three closely related proteins ezrin [67], radixin [68], and moesin [69], that make up the ERM family, along with the related protein merlin [70, 71], belong to the larger 4.1 superfamily of proteins, the members of which are characterized by a FERM (four-point one, ezrin, radixin, moesin) domain at their amino terminus [70, 72-74]. The N-terminal domains of ERM proteins are highly conserved with approximately 85% homology, and are responsible for interacting directly or indirectly with integral membrane proteins [75, 76]. Following the ˜300 residue N-terminal domain is an extended alpha-helical domain followed by an ˜80 residue C-terminal filamentous actin (F-actin) binding domain, which represent the less conserved regions within the ERM proteins [75, 76]. Merlin, the product of the Neurofibromatosis 2 tumor suppressor gene [70, 71], contains an N-terminal FERM domain, shares some of the ERM interactors at the plasma membrane but lacks an F-actin binding domain at its C-terminus[75]. Nevertheless, it too has been reported to interact with F-actin, through actin binding regions within the FERM domain [77, 78].

Consistent with the high degree of structural similarity between ERM proteins is their reported functional redundancy. ERM proteins are co-expressed in cultured cells [66, 76, 79, 83]. The use of constitutively active [84-86] or dominant negative [87-89] versions of the proteins illustrate their crucial roles in cell polarity and morphogenesis. ERM proteins have been implicated in functions ranging from the determination of cell shape, adhesion, motility, cytokinesis, phagocytosis, to integration of membrane transport with signaling cascades [75]. The related protein merlin, shares a number of the aforementioned functions [90].

Regulation of the ERM protein function is thought to occur at the level of their conformation, which then translates to a particular subcellular localization. An inhibitory interaction between the FERM domain and the C-terminal domain maintains ERM proteins as monomers, and confines them to the cytoplasm [91, 92]. Interaction with phosphatidylinositol 4,5-bisphosphate (PIP₂) through the FERM domain followed by phosphorylation at a conserved threonine residue in the C-terminal domain, are thought to unmask both membrane and actin binding sites [93-95]. Inhibitory interactions can occur intermolecularly as well as intramolecularly, however the significance of the oligomeric states is currently unclear [85, 91, 96]. Phosphorylation and phospholipid binding have been proposed to lie downstream of the Rho signaling cascade [93, 97-99], while an ERM function downstream of Rac has also been reported [100]. ERM proteins have also been shown to function upstream Rho, either as positive [101], or as negative [102] regulators, suggesting the presence of a feedback mechanism.

Applicants characterize the novel components of fenestrae sieve plates. In vitro and in vivo evidence was accumulated to support the presence of the ERM protein moesin within fenestrae. The subcellular distribution, phosphorylation state, and detergent solubility of the protein were assessed in the bEND5 cell line. Furthermore, the potential role of moesin in fenestrae biogenesis was addressed through a time-course of fenestrae formation and immunoprecipitation experiments.

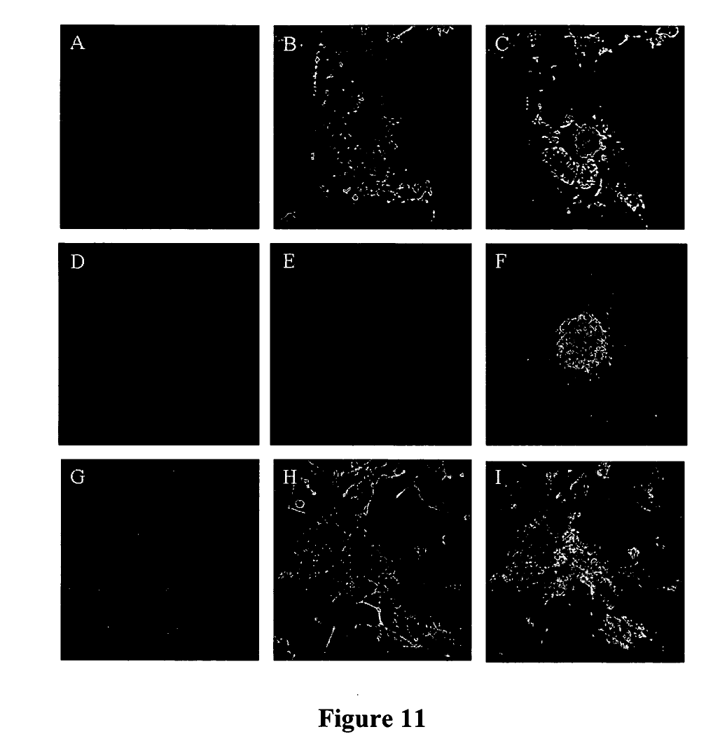

The presence of all ERM family members in most cultured cells, and specifically their co-existence in the cell lines susceptible to fenestrae formation, prompted an investigation into the subcellular distribution of each member (FIG. 11). Latrunculin-induced bEND5 cells were immunostained for ezrin, radixin, moesin, or the related protein merlin. To ascertain for fenestrae localization, anti-PV-1 (MECA-32) was used as a counterstain that marks sieve plates. Despite the reported functional redundancies, the only two ERM members that localized to sieve plates were moesin (FIG. 11, A-C) and radixin (FIG. 11, J-L). In contrast, ezrin (FIG. 11, D-F) and merlin (FIG. 11, G-I), appeared excluded from the sieve plates, and instead were confined to the cytoplasmic arms between them.

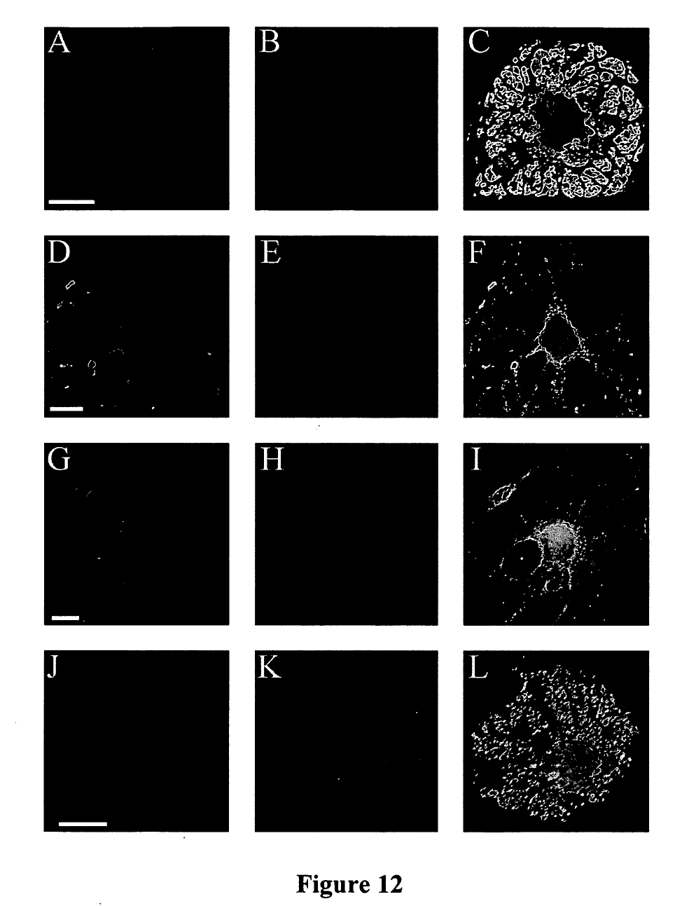

The specificity displayed by ERM family members and merlin for fenestrae in cultured endothelial cells was mirrored in an analysis of a fenestrated capillary bed in vivo. The choriocapillaris of the eye was chosen as a suitable tissue for analysis, as it harbours fenestrae with diaphragms [103, 104] that can be visualized by immunostaining for the diaphragm protein PV-1. In triple stainings of eye sections (FIG. 12), two distinct tissues were immediately visible: the retinal pigment epithelium, with immunoreactivity for ezrin (FIG. 12B,F), as previously reported [105, 106], and the fenestrated choriocapillaris, with immunoreactivity for both PV-1 and moesin (FIG. 12 A, C, E, G). The overlay of all three channels at both low (FIG. 12D), and high (FIG. 12H) magnifications showed colocalization of moesin and PV-1 within the same cell type, and exclusion of ezrin.

Considering its dual binding properties, moesin can act as a coordinator within fenestrae biogenesis, translating the modulation of the cytoskeleton to a remodeling of the plasma membrane. Alternatively, an architectural contribution of moesin in fenestrae sieve plates could involve a role in restricting particular proteins in specialized membrane microdomains, as occurs in the T cell synapse [107] and the podocyte foot processes [115]. Moesin can also play a scaffolding role in providing the cytoskeletal support of the fenestrated area, or in maintaining the close apposition between the apical and basal plasma membranes through its oligomerization properties. Interestingly, the overwhelming majority of specialized cell-surface structures that are associated with ERM proteins share the common property of negative membrane curvature. Intestinal microvilli, stereocilia, podocyte foot processes, and the membrane lining fenestrae, all bear a sharp negative curvature.

In another aspect, the invention provides a method for identifying a plasma membrane marker for fenestrae in an endothelial cell line comprising evolutionary genomics (comparative genomics). With multiple completed genomic sequences available, a comparative genomics approach is used to identify genes that are specifically present in fenestrated organisms. Genes identified from comparative genomics are compared to the proteins identified from proteomics. Common represent gene targets that are highly likely to be involved in the composition or process of forming fenestrae.

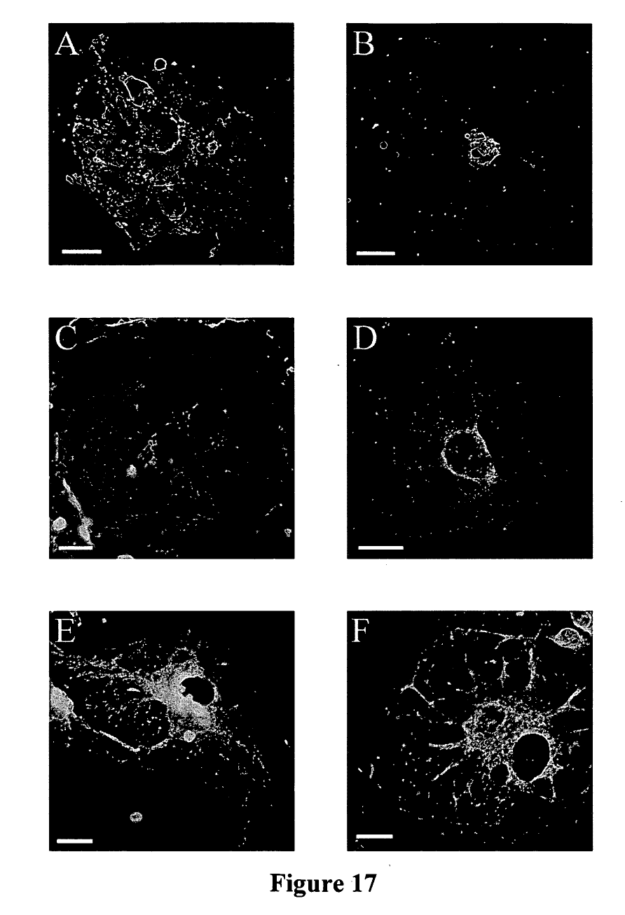

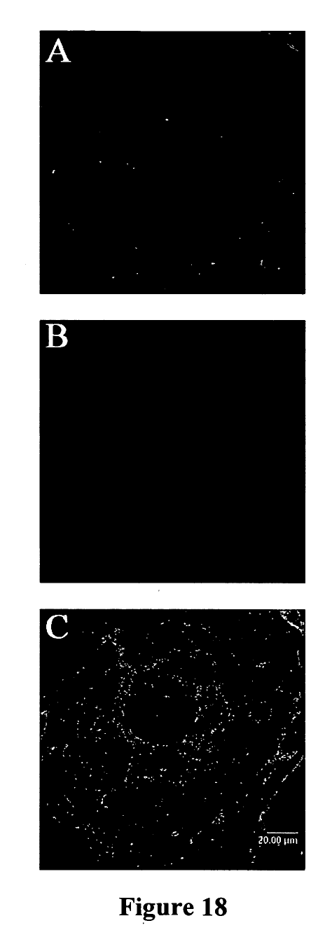

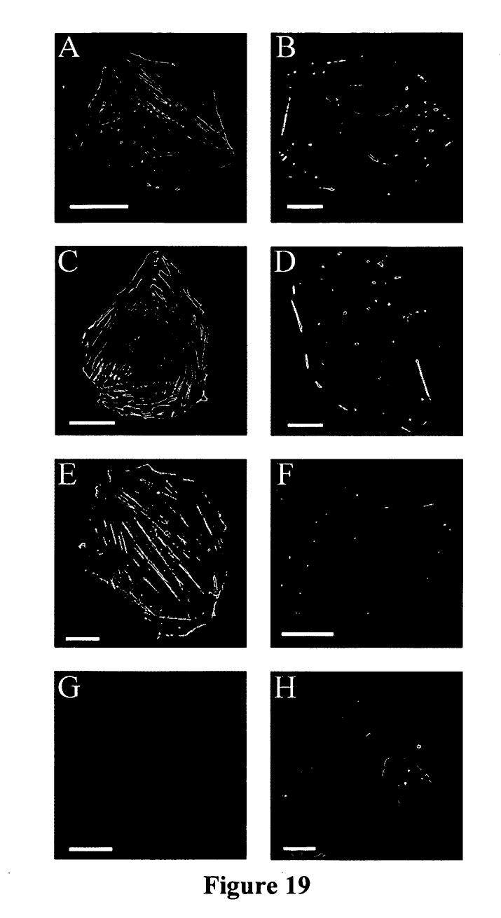

Fenestrae started to appear in organisms that large volume of local fluid or macromolecules exchange are needed. Other than leeches, fenestrae have not been found in any invertebrates (Casley-Smith, J. R. The phylogeny of the fine structure of blood vessels and lymphatics: similarities and differences. Lymphology, 1987. 20: p. 182-188). According to whether fenestrae have appeared, organisms are separated into two classes: fenestrated organisms and non-fenestrated organisms. Fenestrated organisms include human, mouse, rat, zebrafish, and leech. Non-fenestrated organisms include yeast, Arabidopsis, C. elegans, and Drosophila. FIG. 17 shows a Venn Diagram of the comparative approach to enrich for fenestrae-specific genes. The genome of Zebrafish was compared to the genome of human by NCBI-BLASTP to find all matches with a cutoff E value of 10⁻¹⁰. The matches were then compared to the genome of Arabidopsis by NCBI-BLASTP to remove all matches with a cutoff E value of 10⁻¹⁰. The remaining genes are labeled as potential fenestrae-related genes. The remaining genes were consequently compared to genomes of Drosophila, yeast, and C. elegans and matches with a cutoff E value of 10⁻¹⁰ were removed to narrow down the list of genes that are involved in fenestrae. FIG. 18. is a table showing a comparison of fenestrated and non-fenestrated organisms. Musculin, Putative RNA-binding protein 3, Nucleoside diphosphate kinase B, and Paralemmin have been identified both in comparative genomics approach and in proteomics approach (FIG. 19).

In another aspect, the invention relates to a method for altering sieve plate composition. In one embodiment, the method for altering sieve plate composition comprises administering a dominant negative form of moesin. In one particular embodiment, the dominant negative form of moesin is a fusion protein. In another particular embodiment, the dominant negative form of moesin is a fusion protein. comprising a truncated N-terminal domain of moesin fused to Green Fluorescent protein (GFP) (N-moesin/GFP). In another particular embodiment, the dominant negative form of moesin is administered to NIH3T3 cells. In another particular embodiment, the dominant negative form of moesin is administered to bEND5 cells.

In one embodiment, administering the dominant negative form of moesin to bEND5 cells leads to the formation of irregular and branched protrusions that emanate from the cell surface. In another embodiment, administering the dominant negative form of moesin to bEND5 cells leads to the lack of endogenous moesin or endogenous actin.

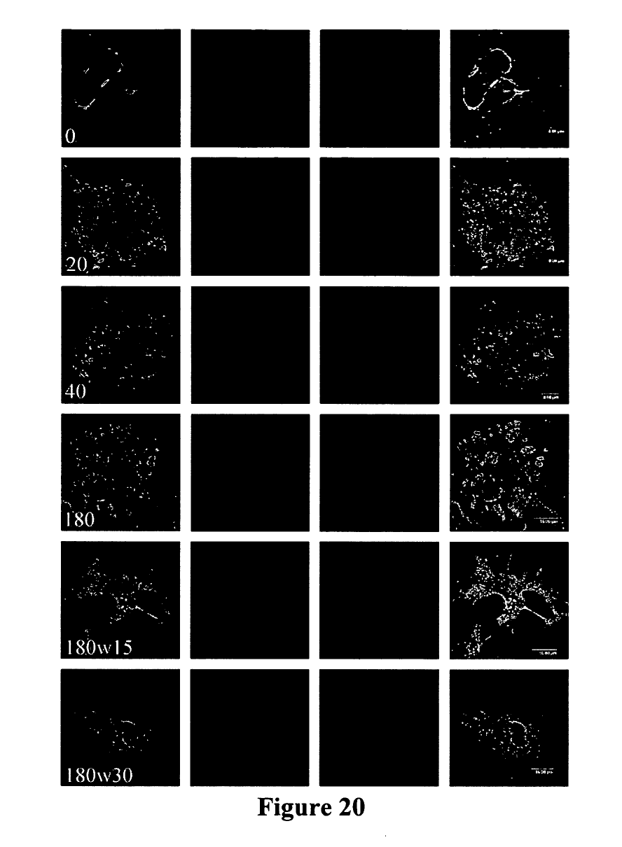

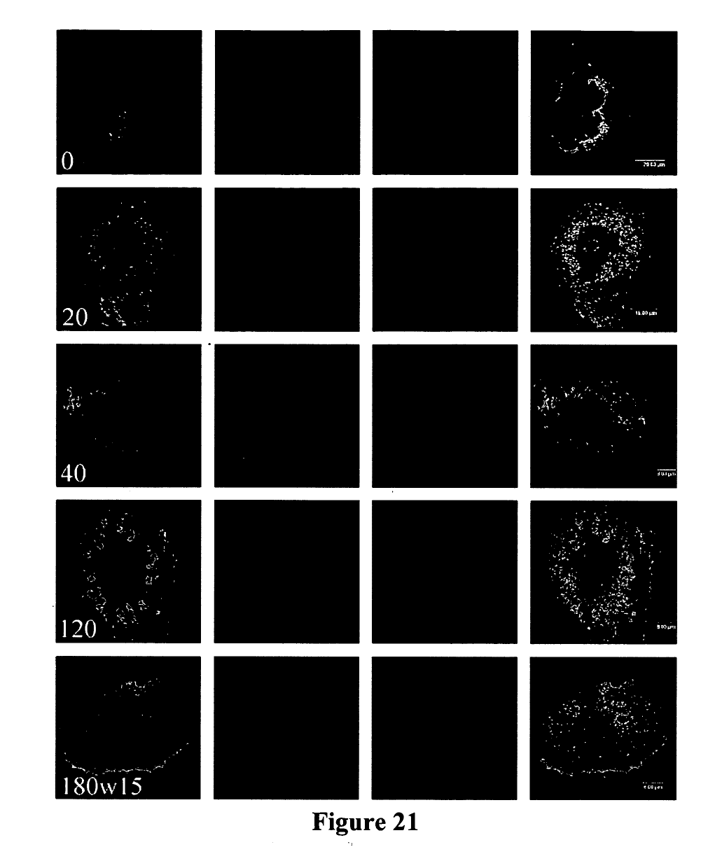

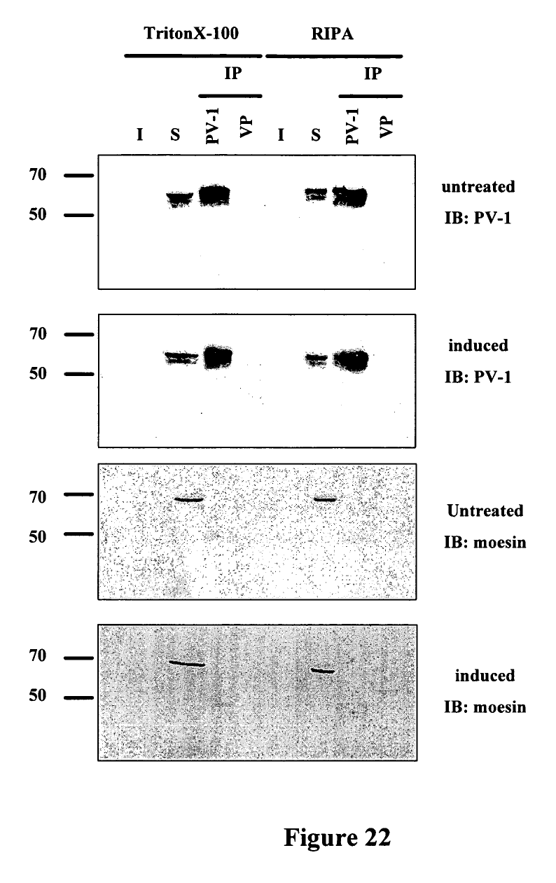

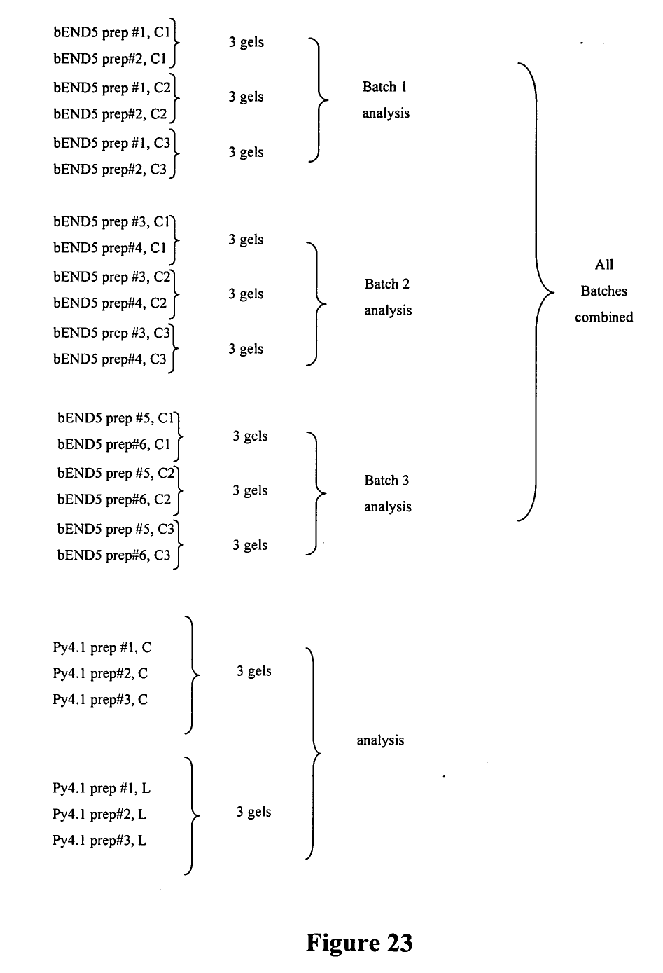

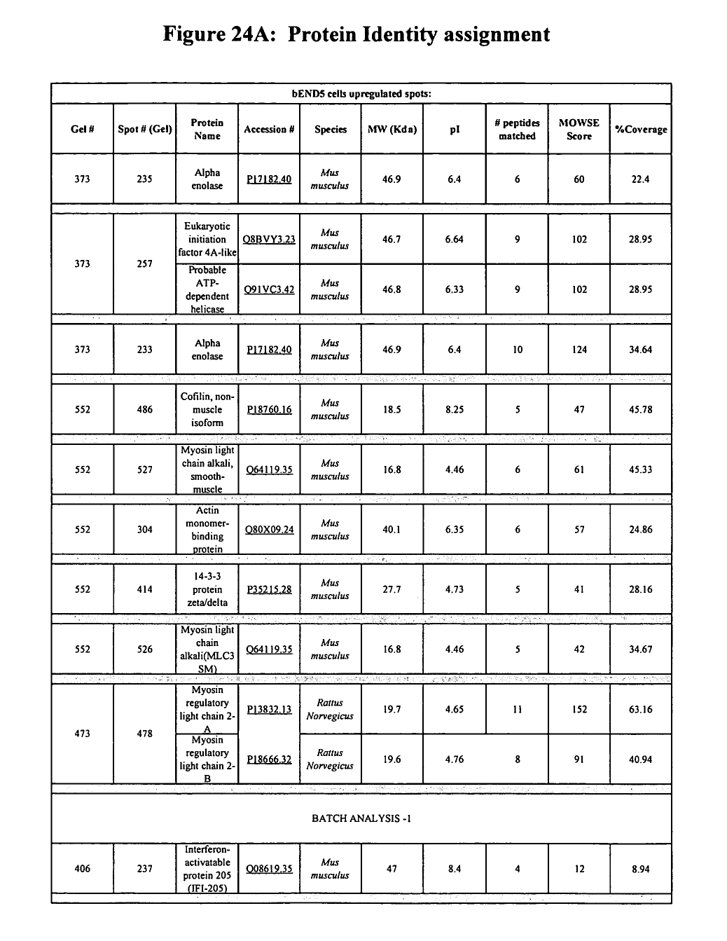

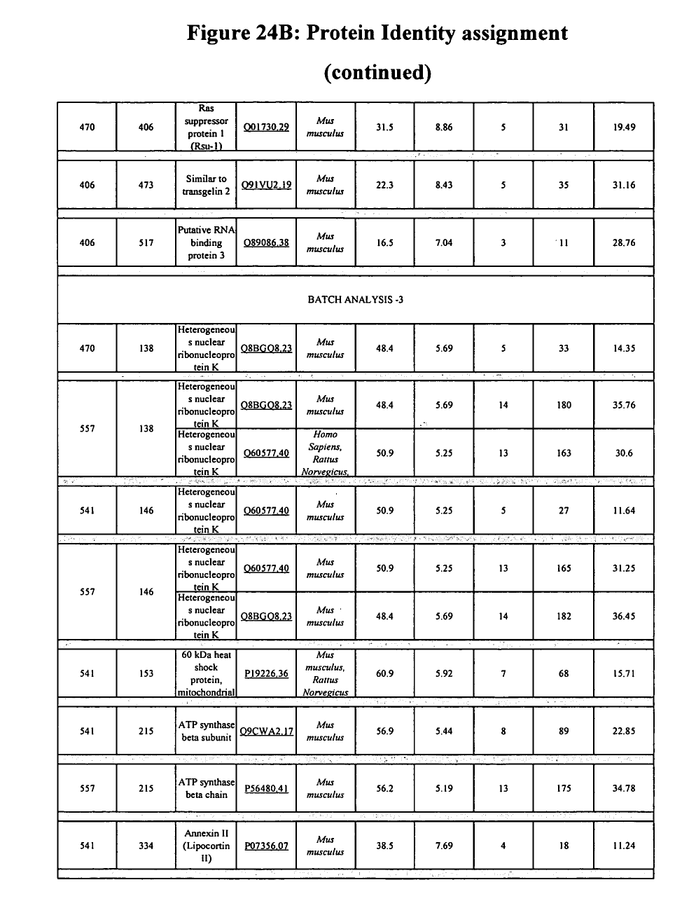

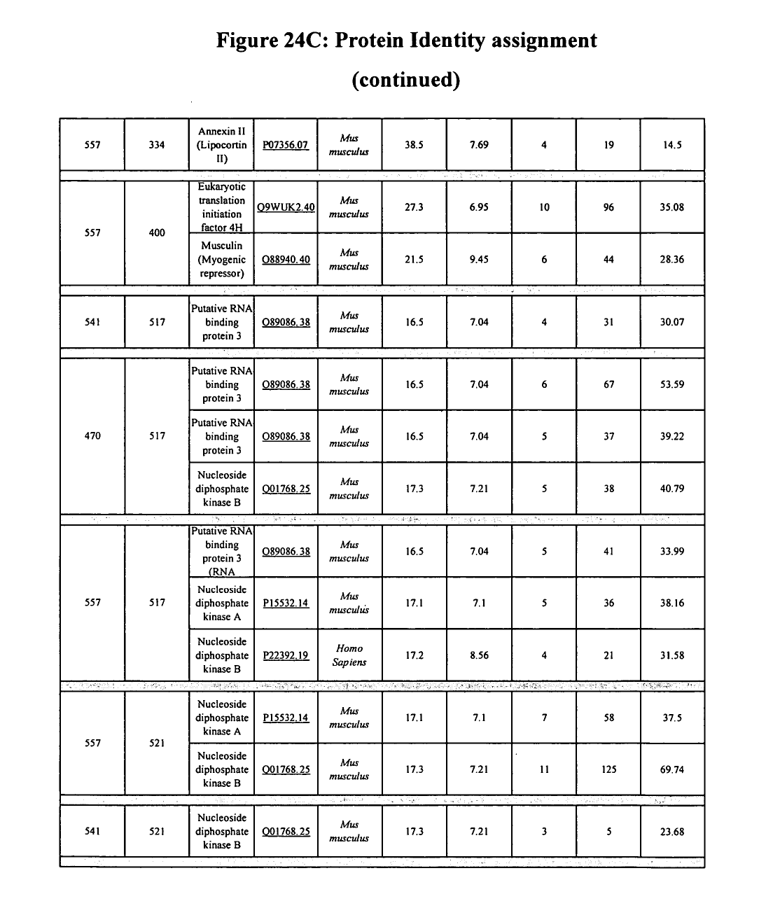

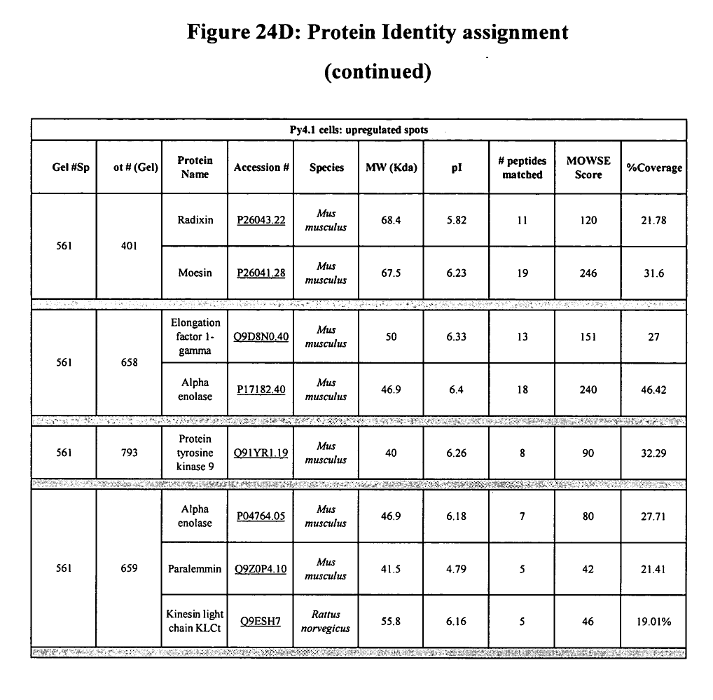

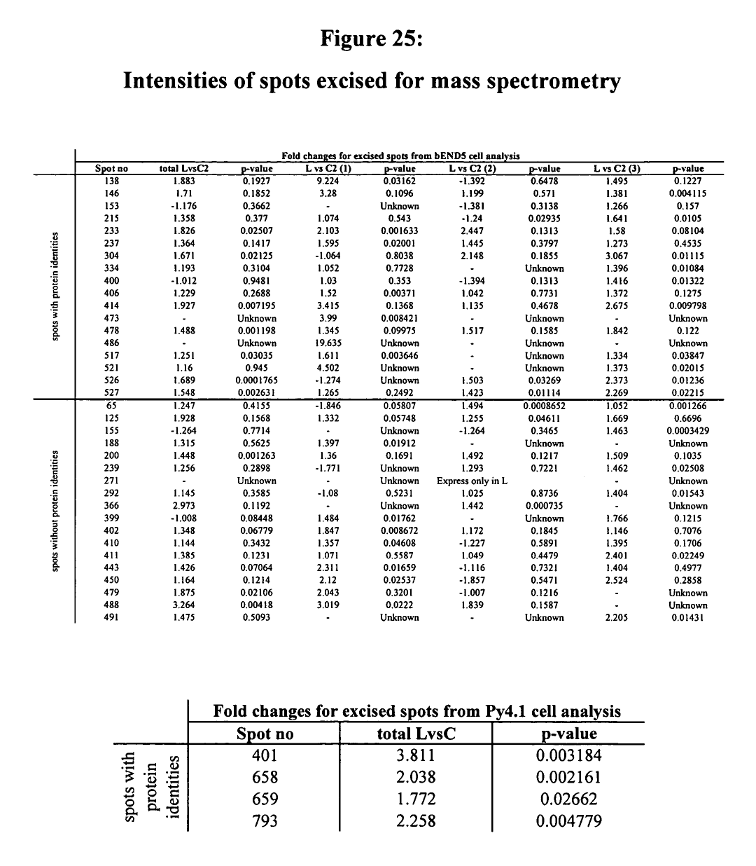

FIG. 20 illustrates a study relating the dominant negative form of moesin (N-moesin/GFP) to sieve plate composition. First, N-moesin/GFP was introduced to induced NIH3T3 or un-induced bEND5 cells. N-moesin/GFP was expressed throughout the plasma membrane and led to the formation of irregular and branched protrusions that emanated from the cell surface. N-moesin/GFP also led to cells lacking endogenous moesin or actin. Second N-moesin/GFP was introduced to bEND5 cells induced with Latrunculin A to form fenestrae and a range of phenotypes was observed by light microscopy, depending on the levels of expression of the fusion protein.

Low-expressing cells formed regular sieve plates, that were encircled by a microtubule-rich border and contained PV-1, moesin, and a discrete network of actin filaments. N-moesin/GFP was expressed throughout the cell surface, coinciding with the presence of PECAM.

High-expressing cells formed sieve plates lacking endogenous moesin (FIG. 20, A-C), PV-1 (FIG. 20, D-F), or a discrete network of actin filaments. N-moesin/GFP was confined to areas of the cell membrane that were morphologically similar to sieve plates (FIG. 20A, 20D, 20G, 20J). The sieve plates occurred in thin areas of the cell periphery, and were distinct from the microtubule- and organelle-rich border that encircled them (FIG. 20J-L). The expression of PECAM throughout the cell surface (FIG. 20, G-I) was unchanged.

FIG. 20M-O illustrates a study concluding that the effect seen by light microscopy was not due to the presence of GFP alone. A GFP-containing plasmid backbone was expressed in bEND5 cells, and fenestrae were induced. In high-expressing induced cells, GFP was distributed throughout the cell cytoplasm, with a preference for microtubule- and organelle-rich regions lying between sieve plates (FIG. 20, M). Moreover, sieve plates presented a regular composition, containing PV-1 (FIG. 20, M-O), moesin and the discrete network of filamentous actin.

Fenestrae are observed in normally non-fenestrated vascular beds in association with unwanted angiogenesis and other cardiovascular pathologies. The appearance of fenestrae in the neovasculature of tumors, retinal vessels in diabetic retinopathy, and capillaries of inflamed tissue, such as arthritic joints, coincides with clinical and experimental findings for vascular leakage and edema and implies that fenestrae contribute to the deregulation of vascular permeability. Fenestrae diameter has also been found to alter in conjunction with liver dysftunction, such as cirrhoses, and in kidney disease, such as pre-eclampsia.

In the first cell biological approach to understanding fenestrae, as described in the present invention herein above, a novel component of fenestrae sieve plates has been characterized. The connection between the membrane-cytoskeleton adaptor moesin and fenestrae bears significant functional implications, and opens new avenues in the study of this organelle's biogenesis. An important step towards the visualization of fenestrae by light microscopy has been accomplished: The classification of fenestrae on the basis of immunoreactivity for PV-1, a marker with dual specificity for caveolae and fenestrae, can be made less ambiguous in combination with anti-moesin labeling, which is specific for the latter. Moreover, moesin immunoreactivity can be useful in probing for non-diaphragmed capillary beds, which lack PV-1, but contain moesin.

### EXAMPLES

The following examples serve to illustrate certain useful embodiments and aspects of the present invention and are not to be construed as limiting the scope thereof. Alternative materials and methods can be utilized to obtain similar results.

Reagents

All chemicals were purchased from Sigma-Aldrich, and Fluka, unless otherwise indicated. Phosphate buffer Saline without calcium or magnesium (PBS), LB medium, LB-agar, EDTA, trypsin/versene, glutamine, penicillin/streptomycin, Leibovitz L-15 medium were provided by CRUK or Eyetech Research Center central services.

Mammalian Tissue Culture

### Example 1

Maintenance of Mammalian Cell Lines

All culture media and related products were obtained from Invitrogen, unless otherwise indicated. Cell lines and culture conditions are shown in Table 3.

TABLE 3

| Cell Line | Species | Origin | Passage No | Culture conditions |
|---|---|---|---|---|
| bEND5 | mouse | brain | 13-25 | DMEM high glucose with sodium pyruvate, |
| | | endothelioma | | 10% FBS, 4 mM L-glutamate, penicilin/streptomycin, |
| | | | | 5 μM β-mercaptoethanol, non-essential amino acids. |
| | | | | 37° C. incubator with 10% CO2 |
| Py4.1 | mouse | ear and tail | | DMEM high glucose with sodium pyruvate, 2% FBS, |
| | | hemangiomas | | penicilin/streptomycin. 37° C. incubator with |
| | | | | 10% CO2 |
| NIH 3T3 | mouse | embryo | | DMEM high glucose with sodium pyruvate, 10% |
| (ATCC) | | | | FBS, 4 mM L-glutamate, penicilin/streptomycin, |
| | | | | 1.5 g/L sodium bicarbonate. 37° C. incubator with |
| | | | | 5% CO2 |
| HUVEC | human | umbilical | 3-5 | M200, low supplement growth serum, |
| (Cascade | | vein | | penicilin/streptomycin (Cascade Biologics). 37° C. |
| Biologics) | | | | incubator with 5% CO2 |
| SVEC4-10 | mouse | lymph node | 3-5 | DMEM high glucose with sodium pyruvate, 10% |
| (ATCC) | | | | FBS, 4 mM L-glutamate, penicilin/streptomycin, |
| | | | | 1.5 g/L sodium bicarbonate. 37° C. incubator with |
| | | | | 10% CO2 |
| MEF | mouse | embryo | 2-3 | DMEM high glucose with sodium pyruvate, 10% |
| (mouse | | | | FBS Tet system approved (Clontech), 4 mM L- |
| embryonic | | | | glutamate, 2× penicilin/streptomycin, 1.25 μg/ml |
| fibroblasts) | | | | Fungizone. 37° C. incubator with 10% CO2 |
| MEF 3T3 Tet- | mouse | embryo | 3 | DMEM high glucose with sodium pyruvate, 10% |
| Off cell line | | | | FBS Tet system approved (Clontech), 4 mM L- |
| (Clontech) | | | | glutamate, penicilin/streptomycin. 37° C. incubator |
| | | | | with 10% CO2 |

All cell lines were trypsinised using trypsin/versene solution. Alternatively cell lines were trypsinized using 1× Trypsin EDTA solution (Invitrogen), apart from bEND5 and Py4.1 cells which required 1× Trypsin EDTA solution (Invitrogen), diluted 1:1 with DMEM.

Cells were thawed by diluting the contents of an ampule in 9 volumes of medium, sedimenting the cells at 300 g and resuspending the pellet in the appropriate volume of complete medium. Cells were frozen in 10% DMSO, 20% Fetal Bovine Serum (FBS), and 70% of complete medium, and were stored in liquid nitrogen.

### Example 2

Fenestrae Induction in Endothelial Cells

Methods for inducing fenestrae formation in endothelial cells are described in U.S. Provisional Patent No. 60/627,981, which is hereby incorporated by reference in its entirety. Coverslips and dishes were coated with 1% gelatin (Sigma) solution in PBS for 30 minutes at room temperature. Endothelial cells were seeded overnight at a density equivalent to 1.5×10⁶ cells per 100 mm dish. Cultures were induced with Cytochalasin B (Sigma) at 10 μM for 2 hours, with Latrunculin A (Molecular Probes) at 2.5 μM for 3 hours, or with a combination of recombinant mouse 75 ng/ml VEGF (R&D systems) for 6-72 hours and 10 μM Cytochalasin B for 2 hours. Cells were processed for biochemistry or morphology immediately after the end of the induction.

To inhibit protein synthesis during fenestrae formation, cells were incubated with 10 μg/ml Cycloheximide (Sigma) for 30 minutes, and then induced with VEGF (75 ng/ml) for 6 hours and Cytochalasin B (10 μM) for the last 2 hours.

Protein Techniques

### Example 3

Protein Concentration Determination

Protein concentrations were determined using the Bio-Rad Protein Assay in microtiter plates. Samples diluted in water, and bovine serum albumin (BSA) standards diluted in water and sample diluent, were incubated with Bio-Rad Protein Assay reagent for 5 minutes at room temperature and the absorbance was measured in a Spectrophotometer at OD₅₉₅. Standard curves were created based on the absorbance of BSA standards and were used to assign protein concentrations to samples. The Detergent Compatible Bio-Rad Protein Assay was used for proteins in buffers containing high concentrations of detergent, and was carried out in a similar fashion, with sample or standard absorbance measured at at OD₇₉₅.

### Example 4

Protein Precipitation

Proteins were precipitated overnight in 80% acetone (v/v) at −80° C. and subsequently centrifuged for 30 minutes at 21000 g at 4° C. The pellet was resuspended in the desired volume of buffer.

### Example 5

Plasma Membrane Isolation Using Cationic Colloidal Silica

All steps in the procedure were performed on ice or in centrifuges pre-cooled to 4° C. Cells grown on 150 mm dishes were washed twice with PBS containing calcium and magnesium (Invitrogen), and once with coating buffer (20 mM MES, 135 mM NaCl, 0.5 mM CaCl₂, 1 mM MgCl₂, pH 5.5). Cells were then coated with 8 ml of a 1% cationic colloidal silica solution (kind gift of Professor Donna Stolz, University of Pittsburg) in coating buffer for 2 minutes. After one wash with coating buffer, cells were overcoated for 1 minute with 8 ml of 1 mg/ml polyacrylic acid (Aldrich) solution in coating buffer, adjusted to pH 6.0-6.5. Cells were washed once with coating buffer and once with lysis buffer (2.5 mM Imidazole, pH 7.0, supplemented with Complete EDTA-free protease inhibitor tablets, Roche), prior to incubation in lysis buffer for 15 minutes. The lysis buffer of induced cells was supplemented with 2.5 μM latrunculin A, to maintain the fenestrated phenotype during the incubation period. Cells were scraped in a total of 2 ml lysis buffer and lysed by 20 strokes in a 7 ml Dounce tissue grinder with a tight pestle (Wheaton). Lysis was ensured by examining the cells by Phase-Contrast Microscopy. Silica-coated plasma membranes and nuclei were sedimented at 900 g for 10 minutes. To obtain internal membranes, the supernatant was centrifuged at 100,000 g for 30 minutes in a TLA 100.3 rotor of an Optima TLX Ultracentrifuge (Beckman Coulter). The plasma membrane and nuclei pellet was resuspended in 1 ml of lysis buffer, supplemented with 20 μl of Benzonase grade II (Merck BDH), and homogenized with 1 ml of 100% Nycodenz in lysis buffer in a dounce tissue grinder. The homogenate was layered on a 0.5 ml cushion of 70% Nycodenz in lysis buffer, and was supplemented with another layer of 2 ml lysis buffer, prior to sedimenting at 60,000 g for 30 minutes in a SW55 Ti rotor of an Optima L-80 Ultracentrifuge (Beckman Coulter). The pellet containing silica-coated plasma membranes was washed 3 times with lysis buffer and membrane proteins were solubilized by boiling silica-coated membranes in silica lysis buffer (3.4% SDS, 120 mM Tris pH 6.8) for 15 minutes. Residual silica was sedimented at 21000 g, and the supernatant containing solubilized plasma membranes was snap-frozen and stored at −80° C. For quality control purposes, samples were removed from various fractions during the course of the purification and were solubilized in silica lysis buffer, treated as the silica-coated plasma membrane pellet. Whole cell lysates were prepared by washing a 150 mm dish twice with PBS, scraping and sedimenting the cells at 300 g, and lysing the pellet in 300 μl silica lysis buffer as above. Equal amounts of solubilized cellular fractions were supplemented with DTT and glycerol (EM Science), to final concentrations of 0.1 M and 5%, respectively, and were separated by SDS-PAGE gel electrophoresis.

### Example 6

Differential Extraction of Cells

Cells grown on 60 mm dishes were briefly washed with PBS and were then extracted for 30 minutes on ice with 0.5 ml of extraction buffer (10 mM Tris pH 8.0, 150 mM NaCl, 2 mM EDTA, Complete EDTA-free Protease inhibitor tablets, Roche, and 0.1%, 0.2%, 0.5%, or 1% Triton X-100, EM Science). Cells were scraped, and insoluble material was pelleted by centrifugation for 30 minutes at 21000 g at 4° C. Detergent soluble material was acetone precipitated, and pellets recovered from either the precipitated soluble material, or from the insoluble material were resuspended in the same volumes of 1×SDS sample buffer, to be separated by SDS-PAGE gel electrophoresis.

### Example 7

Immunoprecipitation

Cells grown on 100 mm dishes were briefly washed with PBS and were then incubated for 30 minutes on ice with 1 ml of immunoprecipitation buffer. The following immunoprecipitation buffers were used:

IP buffer 1: 20 mM Tris HCl pH 7.5, 0.1% Triton X-100 (EM Science), 10% glycerol (EM Science), Complete EDTA-free Protease inhibitor tablets (Roche)

IP buffer 2: 10 mM HEPES pH 7.5, 150 mM NaCl, 0.1% NP-40, 1 mM DTT, Complete EDTA-free Protease inhibitor tablets (Roche)

RIPA buffer: 10 mM Tris pH 7.5, 150 mM NaCl, 1% Triton X-100 (EM Science), 0.5% SDS, 5% sodium deoxycholate, Complete EDTA-free Protease inhibitor tablets(Roche)

Differential extraction buffer: 10 mM Tris pH 8.0, 150 mM NaCl, 2 mM EDTA, Complete EDTA-free Protease inhibitor tablets (Roche), 0.1% Triton X-100(EM Science).

Cells were scraped, and insoluble material was pelleted by centrifugation for 30 minutes at 21000 g at 4° C. Protein concentration of the supernata was measured and 500 μg of the lysate was incubated overnight at 4° C. with 5 μg of antibody. Protein G sepharose™ 4 Fast Flow (Amersham Biosciences) beads were washed 3 times with immunoprecipitation buffer and incubated with the lysate, at a dilution of 1:20 (v/v), for 1 h at 4° C. Immunocomplexes and beads were separated by centrifugation at 16000 g for 5 minutes at 4° C. The beads were washed 3 times with immunoprecipitation buffer and immunocomplexes were eluted by boiling for 10 minutes with 40 μl of 2× SDS sample buffer, to be separated by SDS-PAGE gel electrophoresis.

### Example 8

VI.6. One-Dimensional SDS-Polyacrylamide Gel Electrophoresis

Gels were poured at a thickness of 1 to 1.5 mm using the Bio-Rad Mini gel assembly kit and acrylamide solutions from National Diagnostics. The separating gel was prepared as follows (see table 4):

TABLE 4

| final | | | |
|---|---|---|---|
| acrylamide | | Resolving | deionized H₂O |
| concentration | ProtoGel (ml) | buffer (ml) | (ml) |
| 6% | 2 | 2.5 | 5.39 |
| 8% | 2.67 | 2.5 | 4.72 |
| 10% | 3.33 | 2.5 | 4.06 |
| 12% | 4 | 2.5 | 3.39 |
| 15% | 5 | 2.5 | 2.39 |

100 μl of 10% APS (Sigma) and 10 μl TEMED (Sigma) were added per 10 ml of gel solution to enhance polymerization.

The stacking gel was prepared by mixing 1.3 ml ProtoGel, 2.5 ml ProtoGel Stacking Buffer, 6.1 ml deionized H₂O, and the same amounts of APS and TEMED as above.

SDS sample buffer was prepared as a 6× stock solution, containing 0.35 M Tris HCl, pH 6.8, 10% SDS, 0.6 M DTT, 30% glycerol, and 0.012% bromphenol blue. Samples were boiled in 1× or 2× sample buffer at 95° C. for 5 minutes prior to loading on the gel.

Gels were run in Tris-glycine buffer (25 mM Tris, 250 mM glycine, pH 8.3, 0.1% SDS) at 70-180 V.

### Example 9

Western Blotting

Following electrophoretic separation, proteins were blotted onto Hybond™ (Amersham Biosciences) nitrocellulose membranes using a semi-dry blotter (Bio-Rad). Membranes and pieces of Whatman paper were pre-soaked in transfer buffer (80 ml MeOH, 32 ml Tris-glycine buffer, 0.4 ml of 10% SDS solution, in a total volume of 400 ml) for 20 minutes. After a brief incubation of the gel in transfer buffer, gel and membrane were sandwiched between six pieces of Whatman paper, with the membrane facing the anode of the electroblotting apparatus. Transfer was carried out at 120 mA per gel for 1 hour (1 mm thick gels) or 1.5 hour (1.5 mm thick gels). Blots were incubated with blocking buffer (5% non-fat dry milk in PBS) 2 hours at room temperature or overnight at 4° C. Primary antibody was diluted in blocking buffer and applied for 1-2 hours at room temperature or overnight at 4° C. The following primary antibodies were used: mouse anti-GS28 (1:100; BD Biosciences), rabbit anti-PV1 (1:1000; CRUK), rat anti-MECA-32 (1:500; Developmental Studies Hybridoma Bank), mouse anti-tubulin (1:5000; Sigma), mouse anti-actin (1:2000; clone AC-74; Sigma), mouse anti-caveolin 1 (1:1000; BD Biosciences), goat anti-moesin (1:1000; Santa-Cruz), mouse anti-moesin (1:1000; BD Biosciences), goat anti-radixin (1:1000; Santa-Cruz), mouse anti-ezrin (1:1000; Sigma), rabbit anti-p-Moesin (1:1000; Santa-Cruz), rabbit anti-cofilin (1:1000; Cytoskeleton), mouse anti-annexin II (1:1000; BD Biosciences), goat anti-enolase alpha (1:100; Santa-Cruz), goat anti-hnRNP K (1:100; Santa-Cruz), rabbit anti-merlin (1:1000; gift of Dr. Wallace Ip, University of Cincinnati), mouse anti-GM130 (1:250; BD Biosciences), rat anti-VE-cadherin (1:1000; BD Biosciences). After 3 five-minute washes in blocking buffer, Horseradish peroxidase (HRP) conjugated secondary antibodies were diluted in a similar fashion and applied for 1 hour at room temperature: sheep anti-mouse IgG (1:1000; Amersham Biosciences), donkey anti-rabbit Ig (1:1000; Amersham Biosciences), goat anti-rat (1:1000; Amersham Biosciences), rabbit anti-goat (1:2000; Jackson Immunoresearch). Blots were washed 2 times in blocking buffer and finally 1 time in PBS. HRP reaction product was detected by enhanced chemiluminescence (Amersham Biosciences). Quantitation of the density of bands was performed using Scion Image for Windows (Scion Corporation).

### Example 10

Isoelectric Focusing and Two-Dimensional SDS-PAGE Electrophoresis

Proteins eluted from silica-coated plasma membranes were acetone precipitated and solubilized in 40 mM Tris, 7M Urea, 2M Thiourea, and 1% proprietary detergent, pH 10.4 (Cellular and Organelle Membrane Solubilizing Reagent; Sigma), reduced with tri-butylphosphine, and alkylated with 10 mM acrlamide for 90 minutes at room temperature. Following a second round of acetone precipitation, the pellet was solubilized in 7M Urea, 2M Thiourea, and 2% CHAPS, and 40 μg of protein were subjected to IEF on 11 cm pH 3-10 immobilized pH gradient (IPG) strips (Proteome Systems, Sydney, NSW, Australia). Following IEF, IPG strips were equilibrated in 6M urea, 2% SDS, 50 mM Tris-acetate buffer (pH 7), 0.01% bromophenol blue and subjected to SDS polyacrylamide gel electrophoresis on 6-15% Gel Chips™ (Proteome Systems). All gels were stained in Sypro® Ruby (Molecular Probes) and imaged by CCD camera on a fluorescent imager (Alpha Innotech)

### Example 11

Protein Digestion, MALDI Analysis, and Bioinformatic Database Search

Protein spots were automatically detected and excised using the Xcise apparatus (Shimadzu Biotech). Gel pieces were washed twice with 150 μl 25 mM ammonium bicarbonate, pH 8.2, 50% v/v acetonitrile, then dehydrated by the addition of 100% acetonitrile and air dried. Trypsin (Promega) in 2 mM of 20 μg/mol ammonium bicarbonate was added to each gel piece and incubated at 30° C. for 16 hours. The peptides were extracted by sonication. The solution was automatically desalted and concentrated using Zip Tips (Millipore) on the Xcise apparatus, and spotted onto the Axima (Kratos) MALDI target plate. Peptide mass fingerprints of tryptic peptides were generated by matrix assisted laser desorption/ionization time-of-flight mass spectrometry (MALDI-TOF-MS) using an AximaCFR (Kratos).

Spectra were automatically analyzed by the BioinformatIQ integrated suite of bioinformatics tools (Proteome Systems). Protein identifications were assigned by comparing peak lists to a database containing theoretical tryptic digests of NCBI and Swiss Prot sequence databases. Protein identification was evaluated based on percent coverage, MOWSE score, number of peptide matches, peak intensity, and match of pI and molecular weight with the location of the protein on the 2D gel.

Immunolabeling

### Example 12

Immunohistochemistry on Fixed or Unfixed Mouse Sections

Slides were permeabilized in −20° C. methanol for 10 minutes, and blocked for 30 minutes in blocking solution (10% Donkey or Goat Serum (Sigma), 0.5% Triton X-100, 1% Bovine Serum Albumin (Sigma), PBS) in a humidity chamber. The following primary antibodies were used: goat anti-moesin (1:25; Santa-Cruz), rabbit anti-ezrin (1:100; Upstate), rat anti-MECA-32 (1:100; Developmental Studies Hybridoma Bank), rat anti-mouse CD31 (1:300; clone MEC 13.3; BD Biosciences). 80 μl of primary antibody solution was applied to each slide for 1 hour in a humidity chamber. The slides were washed 3 times, five minutes each time, in blocking solution lacking serum. The same procedure was followed for secondary antibodies Alexa488-conjugated goat anti-rabbit or anti-rat IgG (1:1000; Molecular Probes), Alexa633-conjugated goat anti-rat IgG (1:500; Molecular Probes), and Cy3-conjugated donkey anti-goat IgG (1 μg/ml, Jackson Immunoresearch). As negative controls, the primary antibodies were omitted with secondary antibody incubations performed in the usual way. After three washes, the slides were mounted using vectashield with DAPI (Vector Laboratories Inc.)

### Example 13

Immunofluorescence on Coverslips

Cells on coverslips were fixed either in −20° C. methanol for 8 minutes or in room temperature 4% paraformaldehyde for 15 minutes. For paraformaldehyde fixed cells, fixation was followed by 5 washes in PBS and a 3 minute permeabilization step in PBT (0.1% Triton X-100, PBS). Coverslips were blocked for 15 minutes in blocking solution (10% Goat or Donkey Serum (Sigma), 0.2% Fish Skin Gelatin (Sigma), PBS). Primary antibodies were diluted in blocking solution as follows: rat anti-MECA-32 (1:100; Developmental Studies Hybridoma Bank), mouse anti-tubulin (1:500; Sigma), mouse anti-actin (1:200; clone AC-74; Sigma), mouse anti-caveolin 1 (1:100; BD Biosciences), rabbit anti-caveolin (1:100; BD Biosciences), goat anti-moesin (1:50; Santa-Cruz), mouse anti-moesin (1:100; BD Biosciences), goat anti-radixin (1:25; Santa-Cruz), rabbit anti-ezrin (1:100; Upstate), mouse anti-ezrin (1:100; Sigma), rabbit anti-p-Moesin (1:100; Santa-Cruz), rabbit anti-cofilin (1:100; Cytoskeleton), mouse anti-annexin II (1:100; BD Biosciences), goat anti-enolase alpha (1:100; Santa-Cruz), goat anti-hnRNP K (1:100; Santa-Cruz), goat anti-nm23 H2 (1:100; Santa-Cruz), rabbit anti-merlin (1:100; gift of Dr. Wallace Ip, University of Cincinnati) mouse anti-GM130 (1:100; BD Biosciences). Coverslips were inverted over 50 μl of primary antibody solution on a piece of parafilm, incubated for 30 minutes at room temperature, and finally washed 3 times, five minutes each time, in blocking solution lacking serum. The same procedure was pursued for the following secondary antibodies: Alexa488-conjugated goat anti-rat, anti-mouse, or anti-rabbit IgG (1:1000; Molecular Probes), Alexa633-conjugated goat anti-rat or anti-mouse IgG (1:500; Molecular Probes), Alexa 546-conjugated goat anti-rabbit IgG (1:500; Molecular Probes), Cy3-conjugated donkey anti-goat IgG (1 μg/ml; Jackson Immunoresearch), Cy3-conjugated donkey anti-rat IgG (0.5 μg/ml; Jackson Immunoresearch). To visualize F-actin, Alexa 546-, Alexa 488-, or Alexa 633-conjugated phalloidin (1:50; Molecular Probes) was included in secondary antibody preparations. Alexa 488-conjugated DNase I (1:500; Molecular Probes) was employed to detect G-actin. As negative controls, primary antibodies were omitted with secondary antibody incubations performed in the usual way. After 3 five-minute washes in blocking solution lacking serum, coverslips were mounted on slides using MOWIOL (Calbiochem) with DABCO (Sigma).

### Example 14

Live Cell Labeling for Scanning Electron Microscopy

Cells on coverslips were immediately transferred to ice, and were incubated for 30 minutes with blocking solution (Leibovitz L-15 medium, 0.2% glycine, 1.5% FBS) prior to immunolabeling with primary antib2ody in antibody diluent (L15 medium, 1% BSA, 0.25% Fish Skin Gelatin) for 30. The following primary antibodies were used: rabbit anti-PV1 (1:400; CRUK), rat anti-mouse PECAM (1:100; clone MEC 13.3; BD Biosciences). The cells were washed three times in L15 medium and incubated for 45 minutes with secondary antibodies diluted in antibody diluent as follows: EM goat anti-rabbit IgG 10 nm Gold (1:40; BBInternational), EM goat anti-rat IgG 5 nm Gold (1:40; BBInternational). Following three washes in L15 medium cells were fixed for 2 hours in 3% glutaraldehyde and 1% paraformaldehyde in 0.08 M sodium cacodylate buffer (pH 7.4).

### Example 15

Peptide Competition

Peptide competition of the mouse anti-moesin antibody was performed by incubating the antibody with the peptide against which it was raised (moesin peptide; BD Biosciences) or an irrelevant peptide (SNAP-25; BD Biosciences) at a 10000:1 molar ratio of peptide to antibody. Complexes were incubated for 4-5 hours at room temperature with gentle rotation, and centrifuged at 100000 g for 1 h at 16° C. in a TLA 100.3 rotor of an Optima TLX Ultracentrifuge (Beckman Coulter). The supernatant was used for immunofluorescence as described above.

### Example 16

Light Microscopy

Images were captured using the following instruments and software packages:
1) LSM510 laser scanning confocal microscope (Zeiss); 63×1.40 NA Plan-Achromat oil immersion objective
2) TCS SP2 spectral confocal microscope (Leica); 40×1.25 NA Plan-Achromat oil immersion objective; 63×1.4 NA Plan-Achromat oil immersion objective; 100× . . . ; Leica confocal software version 2.5
3) Widefield DMRA4 microscope (Leica); orca ER2 camera (Hamamatsu); Metamorph Software (Universal Imaging Corporation)
4) MZFL III Fluorescence Stereomicroscope (Leica); Retiga Camera (Q-Imaging); OpenLab 3.1.7 (Improvision, Inc.)
   Digital Images Were Processed Using Adobe Photoshop 7.0 (Adobe Systems Inc.)
   Electron Microscopy

### Example 17

Scanning Electron Microscopy

Cells on coverslips were fixed in 2% paraformaldehyde (EM grade; Electron Microscopy Sciences), 2.5% glutaraldehyde (Sigma), 0.1 M Sodium Cacodylate for 1 hour at room temperature, and maintained in 2% paraformaldehyde, 0.1 M Sodium Cacodylate thereafter. When ready for processing they were washed twice in 0.1 M Sodium Cacodylate, and post-fixed in 1% Osmium Tetraoxide (Agar Scientific Ltd.) in cacodylate buffer. After two washes in distilled water, they were dehydrated through a series of ethanol solutions, 5 minutes in each: 50%, 60%, 70%, 80%, 90%, 100%, 100%. Drying was achieved by immersing in Hexamethyldisalazane (HMDS; Sigma) for 5 minutes, twice, and placing on filter paper.

Cells fixed after live cell labeling were postfixed with 1% aqueous Osmium Tetraoxide for 1 hour, washed once in water, and dehydrated through an ascending ethanol series, 5 minutes in each: 50%, 70%, 90%, 100%, 100%, 100%. Drying was achieved using HMDS as above.

Coverslips were mounted on 10 mm stubs (Agar Scientific Ltd.) using carbon adhesive mounts (Agar Scientific Ltd.). Silver dag (Agar Scientific Ltd.) was added to one edge. The specimens were coated with gold in a Polaron SC7640 High Resolution Sputter Coatter, or carbon in a Gattan 681 Ion Beam Coater, and examined under a JEOL JSM-6700 Field Emmission Scanning EM.

Transmission Electron Microscopy

### Example 18

Wholemount TEM

Formvar grids for wholemount TEM were prepared by coating 400 mesh nickel grids (Gilder; Electron Microscopy Sciences) in a solution of 0.5% Formvar (Ted Pella, Inc.) in ethylene dichloride (Electron Microscopy Sciences). Grids were placed on coverslips, were carbon coated in a BAF 400 D High Vacuum Freeze-Etching System (Balzers) and were UV-sterilized prior to their use in cell culture.

Cells were fixed in 1.25% glutaraldehyde (Electron Microscopy Sciences) and 2.5% paraformaldyde (Electron Microscopy Sciences) in 0.1 M Sodium Cacodylate buffer, for 1 hour at room temperature or overnight at 4° C. Following three five-minute washes in 0.2 M sodium cacodylate cells were postfixed in 1% Osmium tetraoxide (Electron Microscopy Sciences) in cacodylate buffer for 30 minutes. After another 2 washes in 0.2 M sodium cacodylate, and 2 washes in distilled water, cells were dehydrated in an ascending ethanol series. This consisted of one five-minute incubation in 50%, 70% and 85%, ethanol, and two five-minute incubations in 95% and 100% ethanol. Coverslips were immersed in HMDS for 3 minutes and air-dried. Grids were examined under a JEOL 1010 TEM equipped with an AMT digital capture system

### Example 19

TEM of Thin Sections

Agar tubes were prepared by dipping 5 μl glass pipettes into agar (Electron Microscopy Sciences) pre-warmed to 65° C., and cooling them down over ice. Silica-coated plasma membranes were fixed by centrifuging in equal volumes of lysis buffer and 2×EM fix (5% glutaraldehyde, 4% paraformaldehyde, 0.2 M Sodium Cacodylate) at 900 g for 20 minutes at 4° C. Membrane pellets were resuspended and stored in 2% paraformaldehyde, 0.2 M Sodium Cacodylate until ready for processing. Stored membranes were washed twice in 0.2 M Sodium Cacodylate by centrifuging at 900 g for 30 minutes. Membranes were placed into the agar tubes and sealed with warm agar. Tubes with membranes were washed with 0.2 M Sodium Cacodylate for 10 minutes and postfixed with 1% Osmium tetraoxide in 0.1 M Sodium Cacodylated for 1 hour. After two 10—minute washes with 0.2 M Sodium Cacodylate, and one wash with distilled water, specimens were dehydrated through an ascending acetone series of 50%, 70%, 85%, 95%, and 100%. Incubations lasted seven minutes, while 95% and 100% acetone steps were performed twice. Specimens were infiltrated for fourty-five minutes with a mixture of 2 parts acetone and 1 part frozen spurs resin, and then for sixty minutes with a mixture of 1 part acetone and 2 parts frozen spurs resin. Finally, they were infiltrated with 100% fresh spurs resin overnight at room temperature. Tubes were placed into molds and incubated overnight at 60° C. 70-100 nm thick sections,cut using an Ultracut UCT (Leica), were placed onto 200 or 300 mesh copper grids (Gilder; Electron Microscopy Sciences). Grids were stained with Uranyl acetate (Ted Pella, Inc.) for 20 minutes and Reynolds stain (made with Lead Nitrate, Ted Pella, Inc. and Sodium Citrate, Sigma) for 2 minutes prior to examination under the TEM.

### Example 20

Stereology for Electron Microscopy

20 to 30 SEM images were captured at a magnification of 10000. Starting from the centre of the coverslip and systematically random sampling along one direction, an image was captured every 200 μm. Images in which cellular structures occupied less than 50% were dismissed. A grid (2 cm×2 cm squares) was overlaid on each image, and the number of grid points falling on any cellular structure, versus the number of grid points falling on sieve plates (more than 10 fenestrae in a group), were counted. The abundance of fenestrae was estimated using the following formula: fraction of plasma

$$membrane covered with fenestrae = \frac{number of grid points falling on sieve plates}{number of grid points falling on any cell structure}$$

To measure the density of fenestrae, the number of fenestrae per μm² among 10 random sieve plates was calculated; this multiplied by the fraction of plasma membrane covered with fenestrae yielded an estimate of the density of fenestrae per μm² plasma membrane.

For fenestrae quantitation using wholemount TEM, images were captured at a magnification of 25000. 12 images were obtained per nickel grid, and 3 nickel grids were examined per experiment. Images were opened in Adobe Photoshop 7.0, and a 15 mm grid with 169 points was overlaid on each image. The abundance of fenestrae was calculated as in the stereological approach for SEM.

### Example 21

Comparative Genomics

The computer program NCBI-BLASTP with E≦10⁻¹⁰ (Altschul, S. F. et al. Gapped BLAST and PSI-BLAST: a new generation of protein database search programs. Nucleic Acids Res 1997. 25(17): p 3389-3402) was used to generate alignments and employed the blosum 62 amino acid substitution matrix (Henikoff, S. and J. G. Henikoff. Amino acid substitution matrices from peptide blocks. Proc Natl Acad Sci USA, 1992. 89: p 10915-10919). Protein sequence databases of Homo sapiens, Zebrafish, C elegans, Drosophila melanganster were downloaded from Ensembl (Hubbard, T. et al. Ensembl 2005. Nucleic Acids Res. 2005. 33: p D447-D453). These organisms were grouped according to whether or not fenestration was present. All the proteins in human were compared to the proteins in zebrafish using BLASTP with a cutoff E value of 10⁻¹⁰. The comparison provided a data set of 26476 genes. These proteins were then compared to all the proteins in Arabidopsis, yeast, C elegans, and Drosophila. Genes that had a match in non-fenestrated organism were removed from consideration, leaving only the genes both present in fenestrated organisms and absent from non-fenestrated organisms. There were 6661 genes identified representing genes specific to fenestrated organisms. The gene dataset was then compared to the proteins identified through proteomics study. The BLASTP program was used with E value cutoff of 10⁻¹⁰. The following four proteins were identified to be in common between the comparative genomics and proteomics approaches: Musculin, Putative RNA-binding protein 3, Nucleoside diphosphate kinase B, and Paralemmin.

### REFERENCES

1. Stan, R. V., M. Kubitza, and G. E. Palade, PV-1 is a component of the fenestral and stomatal diaphragms in fenestrated endothelia. Proc Natl Acad Sci USA, 1999. 96(23): p. 13203-7.
2. Stan, R. V., Multiple PV1 dimers reside in the same stomatal or fenestral diaphragm. Am J Physiol Heart Circ Physiol, 2004. 286(4): p. H1347-53.
3. Ghitescu, L. D., P. Crine, and B. S. Jacobson, Antibodies specific to the plasma membrane of rat lung microvascular endothelium. Exp Cell Res, 1997. 232(1): p. 47-55.
4. Subcellular Fractionation, in Cells A Laboratory manual. 1998, Cold Spring Harbor Laboratory Press. p. 34. 1-34.9.
5. Jacobson, B. S. and D. Branton, Plasma membrane: rapid isolation and exposure of the cytoplasmic surface by use of positively charged beads. Science, 1977. 195(4275): p. 302-4.
6. Chaney, L. K. and B. S. Jacobson, Coating cells with colloidal silica for high yield isolation of plasma membrane sheets and identification of transmembrane proteins. J Biol Chem, 1983. 258(16): p. 10062-72.
7. Stolz, D. B. and B. S. Jacobson, Examination of transcellular membrane protein polarity of bovine aortic endothelial cells in vitro using the cationic colloidal silica microbead membrane-isolation procedure. J Cell Sci, 1992. 103 (Pt 1): p. 39-51.
8. Mason, P. W. and B. S. Jacobson, Isolation of the dorsal, ventral and intracellular domains of HeLa cell plasma membranes following adhesion to a gelatin substrate. Biochim Biophys Acta, 1985. 821(2): p. 264-76.
9. Simionescu, N., M. Simionescu, and G. E. Palade, Differentiated microdomains on the luminal surface of the capillary endothelium. I. Preferential distribution of anionic sites. J Cell Biol, 1981. 90(3): p. 605-13.
10. Schnitzer, J. E., et al., Separation of caveolae from associated microdomains of GPI-anchored proteins. Science, 1995. 269(5229): p. 1435-9.
11. Schnitzer, J. E., et al., Caveolae from luminal plasmalemma of rat lung endothelium: microdomains enriched in caveolin, Ca(2+)-ATPase, and inositol trisphosphate receptor. Proc Natl Acad Sci USA, 1995. 92(5): p. 1759-63.
12. Rout, M. P., et al., The yeast nuclear pore complex: composition, architecture, and transport mechanism. J Cell Biol, 2000. 148(4): p. 635-51.
13. Garin, J., et al., The phagosome proteome: insight into phagosome functions. J Cell Biol, 2001. 152(1): p. 165-80.
14. Mootha, V. K., et al., Integrated analysis of protein composition, tissue diversity, and gene regulation in mouse mitochondria. Cell, 2003. 115(5): p. 629-40.
15. Andersen, J. S., et al., Proteomic characterization of the human centrosome by protein correlation profiling. Nature, 2003. 426(6966): p. 570-4.
16. Kleffmann, T., et al., The Arabidopsis thaliana chloroplast proteome reveals pathway abundance and novel protein functions. Curr Biol, 2004. 14(5): p. 354-62.
17. Andersen, J. S., et al., Directed proteomic analysis of the human nucleolus. Curr Biol, 2002. 12(1): p. 1-11.
18. Dreger, M., et al., Nuclear envelope proteomics: novel integral membrane proteins of the inner nuclear membrane. Proc Natl Acad Sci USA, 2001. 98(21): p. 11943-8.
19. Bell, A. W., et al., Proteomics characterization of abundant Golgi membrane proteins. J Biol Chem, 2001. 276(7): p. 5152-65.
20. Fialka, I., et al., Subcellular fractionation of polarized epithelial cells and identification of organelle-specific proteins by two-dimensional gel electrophoresis. Electrophoresis, 1997. 18(14): p. 2582-90.
21. Stolz, D. B., G. Bannish, and B. S. Jacobson, The role of the cytoskeleton and intercellular junctions in the transcellular membrane protein polarity of bovine aortic endothelial cells in vitro. J Cell Sci, 1992. 103 (Pt 1): p. 53-68.
22. Oh, P., et al., Subtractive proteomic mapping of the endothelial surface in lung and solid tumours for tissue-specific therapy. Nature, 2004. 429(6992): p. 629-35.
23. Durr, E., et al., Direct proteomic mapping of the lung microvascular endothelial cell surface in vivo and in cell culture. Nat Biotechnol, 2004. 22(8): p. 985-92.
24. Steffan, A. M., J. L. Gendrault, and A. Kim, Increase in the number of fenestrae in mouse endothelial liver cells by altering the cytoskeleton with cytochalasin B. Hepatology, 1987. 7(6): p. 1230-8.
25. Braet, F., et al., Structure and dynamics of the fenestrae-associated cytoskeleton of rat liver sinusoidal endothelial cells. Hepatology, 1995. 21(1): p. 180-9.
26. Braet, F., et al., Microfilament-disrupting agent latrunculin A induces and increased number of fenestrae in rat liver sinusoidal endothelial cells: comparison with cytochalasin B. Hepatology, 1996. 24(3): p. 627-35.
27. Eriksson, A., et al., Small GTP-binding protein Rac is an essential mediator of vascular endothelial growth factor-induced endothelial fenestrations and vascular permeability. Circulation, 2003. 107(11): p. 1532-8.
28. Yokomori, H., et al., Rho modulates hepatic sinusoidal endothelial fenestrae via regulation of the actin cytoskeleton in rat endothelial cells. Lab Invest, 2004. 84(8): p. 1079.
29. Bamburg, J. R., Proteins of the ADF/cofilin family: essential regulators of actin dynamics. Annu Rev Cell Dev Biol, 1999. 15: p. 185-230.
30. Paavilainen, V. O., et al., Regulation of cytoskeletal dynamics by actin-monomer-binding proteins. Trends Cell Biol, 2004. 14(7): p. 386-94.
31. Palmgren, S., M. Vartiainen, and P. Lappalainen, Twinfilin, a molecular mailman for actin monomers. J Cell Sci, 2002. 115(Pt 5): p. 881-6.
32. Lawson, D., M. Harrison, and C. Shapland, Fibroblast transgelin and smooth muscle SM22alpha are the same protein, the expression of which is down-regulated in many cell lines. Cell Motil Cytoskeleton, 1997. 38(3): p. 250-7.
33. Shapland, C., et al., Purification and properties of transgelin: a transformation and shape change sensitive actin-gelling protein. J Cell Biol, 1993. 121(5): p. 1065-73.
34. Tuxworth, R. I. and M. A. Titus, Unconventional myosins: anchors in the membrane traffic relay. Traffic, 2000. 1(1): p. 11-8.
35. Sellers, J. R., Myosins: a diverse superfamily. Biochim Biophys Acta, 2000. 1496(1): p. 3-22.
36. Endow, S. A., Kinesin motors as molecular machines. Bioessays, 2003. 25(12): p. 1212-9.
37. Mundy, D. I., et al., Dual control of caveolar membrane traffic by microtubules and the actin cytoskeleton. J Cell Sci, 2002. 115(Pt 22): p. 4327-39.
38. Gatmaitan, Z., et al., Studies on fenestral contraction in rat liver endothelial cells in culture. Am J Pathol, 1996. 148(6): p. 2027-41.
39. Palacios, F., et al., ARF6-GTP recruits Nm23-H1 to facilitate dynamin-mediated endocytosis during adherens junctions disassembly. Nat Cell Biol, 2002. 4(12): p. 929-36.
40. Kutzleb, C., et al., Paralemmin, a prenyl-palmitoyl-anchored phosphoprotein abundant in neurons and implicated in plasma membrane dynamics and cell process formation. J Cell Biol, 1998. 143(3): p. 795-813.
41. Bretscher, A., K. Edwards, and R. G. Fehon, ERM proteins and merlin: integrators at the cell cortex. Nat Rev Mol Cell Biol, 2002. 3(8): p. 586-99.
42. Lopez-Alemany, R., M. Suelves, and P. Munoz-Canoves, Plasmin generation dependent on alpha-enolase-type plasminogen receptor is required for myogenesis. Thromb Haemost, 2003. 90(4): p. 724-33.
43. Moscato, S., et al., Surface expression of a glycolytic enzyme, alpha-enolase, recognized by autoantibodies in connective tissue disorders. Eur J Immunol, 2000. 30(12): p. 3575-84.
44. de Hoog, C. L., L. J. Foster, and M. Mann, RNA and RNA binding proteins participate in early stages of cell spreading through spreading initiation centers. Cell, 2004. 117(5): p. 649-62.
45. Hegmans, J. P., et al., Proteomic analysis of exosomes secreted by human mesothelioma cells. Am J Pathol, 2004. 164(5): p. 1807-15.
46. Santoni, V., M. Molloy, and T. Rabilloud, Membrane proteins and proteomics: un amour impossible? Electrophoresis, 2000. 21(6): p. 1054-70.
47. Molloy, M. P., et al., Extraction of Escherichia coli proteins with organic solvents prior to two-dimensional electrophoresis. Electrophoresis, 1999. 20(4-5): p. 701-4.
48. Ferro, M., et al., Organic solvent extraction as a versatile procedure to identify hydrophobic chloroplast membrane proteins. Electrophoresis, 2000. 21(16): p. 3517-26.
49. Luche, S., V. Santoni, and T. Rabilloud, Evaluation of nonionic and zwitterionic detergents as membrane protein solubilizers in two-dimensional electrophoresis. Proteomics, 2003. 3(3): p. 249-53.
50. Chevallet, M., et al., New zwitterionic detergents improve the analysis of membrane proteins by two-dimensional electrophoresis. Electrophoresis, 1998. 19(11): p. 1901-9.
51. Pedersen, S. K., et al., Unseen proteome: mining below the tip of the iceberg to find low abundance and membrane proteins. J Proteome Res, 2003. 2(3): p. 303-11.
52. Ferro, M., et al., Integral membrane proteins of the chloroplast envelope: identification and subcellular localization of new transporters. Proc Natl Acad Sci USA, 2002. 99(17): p. 11487-92.
53. Wasiak, S., et al., Enthoprotin: a novel clathrin-associated protein identified through subcellular proteomics. J Cell Biol, 2002. 158(5): p. 855-62.
54. Simpson, R. J., et al., Proteomic analysis of the human colon carcinoma cell line (LIM 1215): development of a membrane protein database. Electrophoresis, 2000. 21(9): p. 1707-32.
55. Galeva, N. and M. Altermann, Comparison of one-dimensional and two-dimensional gel electrophoresis as a separation tool for proteomic analysis of rat liver microsomes: cytochromes P450 and other membrane proteins. Proteomics, 2002. 2(6): p. 713-22.
56. Farsad, K. and P. De Camilli, Mechanisms of membrane deformation. Curr Opin Cell Biol, 2003. 15(4): p. 372-81.
57. Thiele, C., et al., Cholesterol binds to synaptophysin and is required for biogenesis of synaptic vesicles. Nat Cell Biol, 2000. 2(1): p. 42-9.
58. Ostrowski, S. G., et al., Mass spectrometric imaging of highly curved membranes during Tetrahymena mating. Science, 2004. 305(5680): p. 71-3.
59. Musacchio, A., et al., Functional organization of clathrin in coats: combining electron cryomicroscopy and X-ray crystallography. Mol Cell, 1999. 3(6): p. 761-70.
60. Bi, X., R. A. Corpina, and J. Goldberg, Structure of the Sec23/24-Sarl pre-budding complex of the COPII vesicle coat. Nature, 2002. 419(6904): p. 271-7.
61. Takei, K., et al., Tubular membrane invaginations coated by dynamin rings are induced by GTP-gamma S in nerve terminals. Nature, 1995. 374(6518): p. 186-90.
62. Lebel, S. and Y. Raymond, Lamin B from rat liver nuclei exists both as a lamina protein and as an intrinsic membrane protein. J Biol Chem, 1984. 259(5): p. 2693-6.
63. Ferro, M., et al., Proteomics of the chloroplast envelope membranes from Arabidopsis thaliana. Mol Cell Proteomics, 2003.
64. Brugiere, S., et al., The hydrophobic proteome of mitochondrial membranes from Arabidopsis cell suspensions. Phytochemistry, 2004. 65(12): p. 1693-707.
65. Schirmer, E. C., et al., Nuclear membrane proteins with potential disease links found by subtractive proteomics. Science, 2003. 301(5638): p. 1380-2.
66. Sato, N., et al., A gene family consisting of ezrin, radixin and moesin. Its specific localization at actin filament/plasma membrane association sites. J Cell Sci, 1992. 103 (Pt 1): p. 131-43.
67. Bretscher, A., Purification of an 80,000-dalton protein that is a component of the isolated microvillus cytoskeleton, and its localization in nonmuscle cells. J Cell Biol, 1983. 97(2): p. 425-32.
68. Tsukita, S. and Y. Hieda, A new 82-kD barbed end-capping protein (radixin) localized in the cell-to-cell adherens junction: purification and characterization. J Cell Biol, 1989. 108(6): p. 2369-82.
69. Lankes, W., et al., A heparin-binding protein involved in inhibition of smooth-muscle cell proliferation. Biochem J, 1988. 251(3): p. 831-42.
70. Trofatter, J. A., et al., A novel moesin-, ezrin-, radixin-like gene is a candidate for the neurofibromatosis 2 tumor suppressor. Cell, 1993. 72(5): p. 791-800.
71. Rouleau, G. A., et al., Alteration in a new gene encoding a putative membrane-organizing protein causes neuro-fibromatosis type 2. Nature, 1993. 363(6429): p. 515-21.
72. Gould, K. L., et al., cDNA cloning and sequencing of the protein-tyrosine kinase substrate, ezrin, reveals homology to band 4.1. Embo J, 1989. 8(13): p. 4133-42.
73. Lankes, W. T. and H. Furthmayr, Moesin: a member of the protein 4.1-talin-ezrin family of proteins. Proc Natl Acad Sci USA, 1991. 88(19): p. 8297-301.
74. Funayama, N., et al., Radixin is a novel member of the band 4. family. J Cell Biol, 1991. 115(4): p. 1039-48.
75. Bretscher, A., K. Edwards, and R. G. Fehon, ERM proteins and merlin: integrators at the cell cortex. Nat Rev Mol Cell Biol, 2002. 3(8): p. 586-99.
76. Tsukita, S. and S. Yonemura, Cortical actin organization: lessons from ERM (ezrin/radixin/moesin) proteins. J Biol Chem, 1999. 274(49): p. 34507-10.
77. Xu, H. M. and D. H. Gutmann, Merlin differentially associates with the microtubule and actin cytoskeleton. J Neurosci Res, 1998. 51(3): p. 403-15.
78. Brault, E., et al., Normal membrane localization and actin association of the NF2 tumor suppressor protein are dependent on folding of its N-terminal domain. J Cell Sci, 2001. 114(Pt 10): p. 1901-12.
79. Berryman, M., Z. Franck, and A. Bretscher, Ezrin is concentrated in the apical microvilli of a wide variety of epithelial cells whereas moesin is found primarily in endothelial cells. J Cell Sci, 1993. 105 (Pt 4): p. 1025-43.
80. Schwartz-Albiez, R., et al., Differential expression of the microspike-associated protein moesin in human tissues. Eur J Cell Biol, 1995. 67(3): p. 189-98.
81. Doi, Y., et al., Normal development of mice and unimpaired cell adhesion/cell motility/actin-based cytoskeleton without compensatory up-regulation of ezrin or radixin in moesin gene knockout. J Biol Chem, 1999. 274(4): p. 2315-21.
82. Kitajiri, S., et al., Radixin deficiency causes deafness associated with progressive degeneration of cochlear stereocilia. J Cell Biol, 2004. 166(4): p. 559-70.
83. Amieva, M. R. and H. Furthmayr, Subcellular localization of moesin in dynamic filopodia, retraction fibers, and other structures involved in substrate exploration, attachment, and cell-cell contacts. Exp Cell Res, 1995. 219(1): p. 180-96.
84. Oshiro, N., Y. Fukata, and K. Kaibuchi, Phosphorylation of moesin by rho-associated kinase (Rho-kinase) plays a crucial role in the formation of microvilli-like structures. J Biol Chem, 1998. 273(52): p. 34663-6.
85. Gautreau, A., D. Louvard, and M. Arpin, Morphogenic effects of ezrin require a phosphorylation-induced transition from oligomers to monomers at the plasma membrane. J Cell Biol, 2000. 150(1): p. 193-203.
86. Pujuguet, P., et al., Ezrin regulates E-cadherin-dependent adherens junction assembly through Racl activation. Mol Biol Cell, 2003. 14(5): p. 2181-91.
87. Amieva, M. R., et al., Disruption of dynamic cell surface architecture of NIH3T3 fibroblasts by the N-terminal domains of moesin and ezrin: in vivo imaging with GFP fusion proteins. J Cell Sci, 1999. 112 (Pt 1): p. 111-25.
88. Crepaldi, T., et al., Ezrin is an effector of hepatocyte growth factor-mediated migration and morphogenesis in epithelial cells. J Cell Biol, 1997. 138(2): p. 423-34.
89. Allenspach, E. J., et al., ERM-dependent movement of CD43 defines a novel protein complex distal to the immunological synapse. Immunity, 2001. 15(5): p. 739-50.
90. Bretscher, A., et al., ERM-Merlin and EBP50 protein families in plasma membrane organization and function. Annu Rev Cell Dev Biol, 2000. 16: p. 113-43.
91. Berryman, M., R. Gary, and A. Bretscher, Ezrin oligomers are major cytoskeletal components of placental microvilli: a proposal for their involvement in cortical morphogenesis. J Cell Biol, 1995. 131(5): p. 1231-42.
92. Bretscher, A., R. Gary, and M. Berryman, Soluble ezrin purified from placenta exists as stable monomers and elongated dimers with masked C-terminal ezrin-radixin-moesin association domains. Biochemistry, 1995. 34(51): p. 16830-7.
93. Matsui, T., et al., Rho-kinase phosphorylates COOH-terminal threonines of ezrin/radixin/moesin (ERM) proteins and regulates their head-to-tail association. J Cell Biol, 1998. 140(3): p. 647-57.
94. Simons, P. C., et al., C-terminal threonine phosphorylation activates ERM proteins to link the cell's cortical lipid bilayer to the cytoskeleton. Biochem Biophys Res Commun, 1998. 253(3): p. 561-5.
95. Fievet, B. T., et al., Phosphoinositide binding and phosphorylation act sequentially in the activation mechanism of ezrin. J Cell Biol, 2004. 164(5): p. 653-9.
96. Gary, R. and A. Bretscher, Heterotypic and homotypic associations between ezrin and moesin, two putative membrane-cytoskeletal linking proteins. Proc Natl Acad Sci USA, 1993. 90(22): p. 10846-50.
97. Fukata, Y., et al., Association of the myosin-binding subunit of myosin phosphatase and moesin: dual regulation of moesin phosphorylation by Rho-associated kinase and myosin phosphatase. J Cell Biol, 1998. 141(2): p. 409-18.
98. Shaw, R. J., et al., RhoA-dependent phosphorylation and relocalization of ERM proteins into apical membrane/actin protrusions in fibroblasts. Mol Biol Cell, 1998. 9(2): p. 403-19.
99. Kotani, H., et al., Rho regulates association of both the ERM family and vinculin with the plasma membrane in MDCK cells. Oncogene, 1997. 14(14): p. 1705-13.
100. Mackay, D. J., et al., Rho- and rac-dependent assembly of focal adhesion complexes and actin filaments in permeabilized fibroblasts: an essential role for ezrin/radixin/moesin proteins. J Cell Biol, 1997. 138(4): p. 927-38.
101. Takahashi, K., et al., Direct interaction of the Rho GDP dissociation inhibitor with ezrin/radixin/moesin initiates the activation of the Rho small G protein. J Biol Chem, 1997. 272(37): p. 23371-5.
102. Speck, O., et al., Moesin functions antagonistically to the Rho pathway to maintain epithelial integrity. Nature, 2003. 421(6918): p. 83-7.
102. Pino, R. M., Restriction to endogenous plasma proteins by a fenestrated capillary endothelium: an ultrastructural immunocytochemical study of the choriocapillary endothelium. Am J Anat, 1985. 172(4): p. 279-89.
104. Guymer, R. H., A. C. Bird, and G. S. Hageman, Cytoarchitecture of choroidal capillary endothelial cells. Invest Ophthalmol Vis Sci, 2004. 45(6): p. 1660-6.
105. Hofer, D. and D. Drenckhahn, Molecular heterogeneity of the actin filament cytoskeleton associated with microvilli of photoreceptors, Muller's glial cells and pigment epithelial cells of the retina. Histochemistry, 1993. 99(1): p. 29-35.
106. Bonilha, V. L., S. C. Finnemann, and E. Rodriguez-Boulan, Ezrin promotes morphogenesis of apical microvilli and basal infoldings in retinal pigment epithelium. J Cell Biol, 1999. 147(7): p. 1533-48.
107. Delon, J., K. Kaibuchi, and R. N. Germain, Exclusion of CD43 from the immunological synapse is mediated by phosphorylation-regulated relocation of the cytoskeletal adaptor moesin. Immunity, 2001. 15(5): p. 691-701.
108. Tsukita, S., et al., ERM family members as molecular linkers between the cell surface glycoprotein CD44 and actin-based cytoskeletons. J Cell Biol, 1994. 126(2): p. 391-401.
109. Vanni, C., et al., Phosphorylation-independent membrane relocalization of ezrin following association with Dbl in vivo. Oncogene, 2004. 23(23): p. 4098-106.
110. Franck, Z., R. Gary, and A. Bretscher, Moesin, like ezrin, colocalizes with actin in the cortical cytoskeleton in cultured cells, but its expression is more variable. J Cell Sci, 1993. 105 (Pt 1): p. 219-31.
111. Serrador, J. M., et al., Moesin interacts with the cytoplasmic region of intercellular adhesion molecule-3 and is redistributed to the uropod of T lymphocytes during cell polarization. J Cell Biol, 1997. 138(6): p. 1409-23.
112. Stickney, J. T., et al., Activation of the tumor suppressor merlin modulates its interaction with lipid rafts. Cancer Res, 2004. 64(8): p. 2717-24.
113. Amieva, M. R., K. K. Wilgenbus, and H. Furthmayr, Radixin is a component of hepatocyte microvilli in situ. Exp Cell Res, 1994. 210(1): p. 140-4.
114. Hugo, C., et al., The plasma membrane-actin linking protein, ezrin, is a glomerular epithelial cell marker in glomerulogenesis, in the adult kidney and in glomerular injury. Kidney Int, 1998. 54(6): p. 1934-44.
115. Takeda, T., et al., Loss of glomerular foot processes is associated with uncoupling of podocalyxin from the actin cytoskeleton. J Clin Invest, 2001. 108(2): p. 289-301.
   Incorporation by Reference

The patent and scientific literature referred to herein establishes knowledge that is available to those of skill in the art. All issued patents, patent applications, published foreign applications, and published references, which are cited herein, are hereby incorporated by reference to the same extent as if each was specifically and individually indicated to be incorporated by reference in their entirety.

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A method of identifying a marker for fenestrae in an endothelial cell line comprising the steps of:
a) isolating plasma membranes from a batch of fenestrae induced endothelial cells and a batch of fenestrae un-induced endothelial cells.
b) running a two-dimensional electrophoresis gel on material isolated from the batch of fenestrae induced endothelial cells;
c) running a two-dimensional electrophoresis gel on material isolated from the batch of fenestrae un-induced endothelial cells;
d) determining fold differences between staining intensities of spots in the two-dimensional gel of the batch of fenestrae induced endothelial cells and the spots in the two-dimensional gel of the batch of fenestrae un-induced endothelial cells;
e) excising a spot; and
f) analyzing the spot against a database of fingerprints from theoretical tryptic digests of proteins so as to assign a particular protein identity to the spot.

2. The method of claim 1, further comprising the step of producing a characteristic mass fingerprint for the excised spot utilizing mass spectrometry.

3. The method of claim 1, further comprising the step of confirming the assigned protein identity of the spot.

4. The method of claim 2, wherein the step of confirming the protein identity of the spot comprises immunoblotting antibodies onto the relevant fractions of candidate proteins.

5. The method of claim 2, wherein the step of confirming the findings of the spot comprises immunofluorescence against a characteristic pattern of PV-1 in induced cells as a reference standard.

6. The method of claim 1, wherein the endothelial cell line is selected from the group consisting of a bEND5 endothelial cell line and a Py4.1 endothelial cell line.

7. The method of claim 1, wherein the endothelial cell line is a brain endothelioma cell line.

8. The method of claim 1, wherein the step of isolating plasma membranes comprises a silica isolation procedure.

9. A method for visualizing fenestrae in an endothelial cell line, said method comprising the steps of:
a) staining an endothelial cell line with an anti-moesin antibody;
b) imaging the endothelial cell line utilizing a light microscope; and
c) visualizing fenestrae based on the level of the fluorescently labeled anti-moesin bound to moesin in the endothelial cell line.

10. The method of claim 9, wherein the anti-moesin antibody is fluorescently labeled.

11. The method of claim 9, wherein the endothelial cell line is selected from the group consisting of a bEND5 endothelial cell line and a Py4.1 endothelial cell line.

12. The method of claim 9, wherein the endothelial cell line is a brain endothelioma cell line.

13. A marker for detecting fenestrae in an endothelial cell line, wherein the marker comprises a protein selected from the group consisting of moesin, paralemmin, radixin, cofilin, twinfilin, alpha-enolase, annexin II, musculin, putative RNA-binding protein 3, nucleoside diphosphate kinase B, and hnRNP K.

14. The marker of claim 13, wherein the marker is a plasma membrane marker for fenestrae.

15. The marker of claim 13, wherein the marker comprises a protein selected from the group consisting of moesin and radixin.

16. A marker combination for detecting fenestrae, wherein the marker combination comprises moesin and PV-1.

17. A method for identifying fenestrae in an endothelial cell line comprising the step of visualizing a marker combination comprising moesin and PV-1.

18. The method of claim 17, wherein the step of visualizing a marker combination comprising moesin and PV-1 comprises light microscopy.

19. The method of claim 17, wherein the endothelial cell line is selected from the group consisting of a bEND5 endothelial cell line and a Py4.1 endothelial cell line.

20. The method of claim 17, wherein the endothelial cell line is a brain endothelioma cell line.

21. A method of identifying fenestrae in an endothelial cell line comprising the step of detecting PV-1 and paralemmin.

22. A method of identifying a marker for fenestrae in an endothelial cell line comprising the step of performing subtractive proteomic analysis.

23. The method of claim 22, further comprising the step of performing evolutionary genomics.

24. A method of identifying a marker for fenestrae in an endothelial cell line comprising the step of performing evolutionary genomics.

25. A method of altering sieve plate composition in a fenestrated cell line comprising the step of administering a dominant negative version of moesin to the fenestrated cell line.

26. The method of claim 26, wherein the fenestrated cell line is selected from the group consisting of a NIH3T3cell line and a bEND5 cell line.

27. The method of claim 26, wherein the dominant negative version of moesin is a fusion protein.

28. The method of claim 26, wherein the dominant negative version of moesin is a fusion protein comprising an N-terminal domain of moesin fused to a Green Fluorescent Protein.
